(19) United States
(12) Patent Application Publication
(10) Pub. No.: US 2004/0058437 A1
(43) Pub. Date: Mar. 25, 2004

(54) Materials and reactor systems having humidity and gas control

(76) Inventors: Seth T. Rodgers, Somerville, MA (US); A. Peter Russo, Woburn, MA (US); Howard B. Schreyer, Tewksbury, MA (US); Andrey J. Zarur, Winchester, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/457,049

(22) Filed: Jun. 5, 2003

Related U.S. Application Data
(63) Continuation-in-part of application No. 10/119,917, filed on Apr. 10, 2002.
(60) Provisional application No. 60/386,323, filed on Jun. 5, 2002.
(60) Provisional application No. 60/282,741, filed on Apr. 10, 2001.

(51) Int. Cl. C12M 1/12

(52) U.S. Cl. 435/297.1; 435/297.5; 435/325

(57) ABSTRACT

The present invention is directed to materials and reactor systems having humidity and/or gas control. The material may have high oxygen permeability and/or low water vapor permeability. In some cases, the material may have sufficient permeance and/or permeability to allow cell culture to occur in a chip or other reactor system using the material. In certain embodiments, the material may be positioned adjacent to or abut a reaction site within a chip or reactor; in other embodiments, the material may be positioned such that it is in fluidic communication with the reaction site. The material may also be porous and/or transparent in some cases. In one set of embodiments, the material include a polymer that is branched, and/or contains bulky side groups that allow the polymer to have a more open structure. In some cases, the material may include two or more layers. Each layer may have a desired property, which may include, for example, permeability, transparency, cytophilicity, biophilicity, hydrophilicity, or a structural feature. In some embodiments, the material may be chosen so as to promote cell growth within the chip or reactor.

### RELATED APPLICATIONS

This application claims the benefit under 35 U.S.C. 119(e) of co-pending U.S. Provisional Patent Application Serial No. 60/386,323, filed Jun. 5, 2002, entitled “Materials and Reactors having Humidity and Gas Control,” by S. Rodgers, et al., incorporated herein by reference in its entirety. This application is also a continuation-in-part of co-pending U.S. patent application Ser. No. 10/119,917, filed Apr. 10, 2002, entitled “Microfermentor Device and Cell Based Screening Method,” by A. Zarur, et al., which claims priority to U.S. Provisional Patent Application Serial No. 60/282,741, filed Apr. 10, 2001.

### FIELD OF THE INVENTION

The present invention is directed to materials and reactor systems having humidity and gas control.

### BACKGROUND OF INVENTION

A wide variety of reaction systems are known for the production of products of chemical and/or biochemical reactions. Chemical plants involving catalysis, biochemical fermenters, pharmaceutical production plants, and a host of other systems are well-known. Biochemical processing may involve the use of a live microorganism (e.g., cells) to produce a substance of interest.

Cells are cultured for a variety of reasons. Increasingly, cells are cultured for proteins or other valuable materials they produce. Many cells require specific conditions, such as a controlled environment. The presence of nutrients, metabolic gases such as oxygen and/or carbon dioxide, humidity, as well as other factors such as temperature, may affect cell growth. Cells require time to grow, during which favorable conditions must be maintained. In some cases, such as with particular bacterial cells, a successful cell culture may be performed in as little as 24 hours. In other cases, such as with particular mammalian cells, a successful culture may require about 30 days or more.

Typically, cell cultures are performed in media suitable for cell growth and containing necessary nutrients. The cells are generally cultured in a location, such as an incubator, where the environmental conditions can be controlled. Incubators traditionally range in size from small incubators (e.g., about 1 cubic foot) for a few cultures up to an entire room or rooms where the desired environmental conditions can be carefully maintained.

Recently, as described in International Patent Application Serial No. PCT/US01/07679, published on Sep. 20, 2001 as WO 01/68257, entitled “Microreactors,” incorporated herein by reference, cells have also been cultured on a very small scale (i.e., on the order of a few milliliters or less), so that, among other things, many cultures can be performed in parallel.

### SUMMARY OF THE INVENTION

The present invention is directed to materials and reactor systems having humidity and gas control. In some cases, the reactor systems may be used to maintain living cells for relatively long periods of time. A variety of reactor systems are provided, as well as methods involving the use of such materials and reactor systems. The subject matter of this application involves, in some cases, interrelated products and/or uses, alternative solutions to a particular problem, and/or a plurality of different uses of a single system or article.

In one aspect, the invention includes a membrane. The membrane, in one set of embodiments, includes a material having a permeability to oxygen greater than about 50 (cm³_{STP} mm/m² atm day) and a permeability to water vapor lower than about 6×10⁶ (cm³_{STP} mm/m² atm day). In another set of embodiments, the membrane includes a first layer comprising at least 55% by weight of a first polymer or copolymer, a second layer comprising no more than 45% by weight of the first polymer or copolymer, a permeability to oxygen greater than about 1×10² (cm³_{STP} mm/m² atm day), and a permeability to water vapor lower than about 6×10⁶ (cm³_{STP} mm/m² atm day).

In another aspect, the invention includes an apparatus. In one set of embodiments, the apparatus includes a chip comprising a predetermined reaction site including a membrane comprising a permeability to oxygen greater than about 1×10² (cm³_{STP} mm/m² atm day) and a permeability to water vapor lower than about 6×10⁶ (cm³_{STP} mm/m² atm day). The apparatus, in another set of embodiments, includes a chip comprising a predetermined reaction site no greater than 1 milliliter in volume, and a humidity controller positioned adjacent to the predetermined reaction site. The apparatus includes, in yet another set of embodiments, a chip comprising a predetermined reaction site no greater than 1 milliliter in volume, and a humidity controller having sufficient oxygen permeability and a low water vapor permeability selected to allow cell growth within the predetermined reaction site. In still another set of embodiments, the apparatus includes a chip comprising a predetermined reaction site no greater than 1 milliliter in volume, and a humidity controller able to maintain a humidity level and an oxygen concentration in the predetermined reaction site sufficient to allow cell growth within the predetermined reaction site. In some embodiments, the apparatus includes a chip comprising a predetermined reaction site no greater than 1 milliliter in volume, and a humidity controller positioned adjacent to the predetermined reaction site. The apparatus, in certain embodiments, includes a chip comprising a predetermined reaction site no greater than 1 milliliter in volume, and a humidity controller having sufficient oxygen permeability and a low water vapor permeability selected to allow cell growth within the predetermined reaction site. In certain embodiments, the apparatus includes a chip comprising a predetermined reaction site no greater than 1 milliliter in volume, and a gas-permeable surface positioned adjacent to the predetermined reaction site.

In one set of embodiments, the apparatus includes a chip comprising a predetermined reaction site no greater than 1 milliliter in volume; and a humidity controller positioned adjacent to the predetermined reaction site. The apparatus, in another set of embodiments, includes a chip comprising a predetermined reaction site no greater than 1 milliliter in volume; and a humidity controller having sufficient oxygen permeability and a low water vapor permeability selected to allow cell growth within the predetermined reaction site. In yet another set of embodiments, the apparatus includes a chip comprising a predetermined reaction site no greater than 1 milliliter in volume, and a humidity controller able to maintain a humidity level and an oxygen concentration in the reaction site sufficient to allow cell growth within the predetermined reaction site. In still another set of embodiments, the apparatus includes a chip comprising a predetermined reaction site no greater than 1 milliliter in volume, and a gas-permeable surface positioned adjacent to the predetermined reaction site.

The invention is defined by a method in another aspect. In one set of embodiments, the method is a method of culturing cells. The method includes the steps of identifying an oxygen requirement and a humidity requirement of the cells, selecting a material having an oxygen permeability high enough to meet the oxygen requirement of the cells and a water vapor permeability low enough to meet the humidity requirement of the cells, and culturing the cells in a chip comprising a predetermined reaction site having a volume of no more than 1 milliliter and at least one wall having at least a portion thereof formed of the material.

In one embodiment, the invention includes a method comprising the steps of providing a chip comprising a predetermined reaction site no greater than 1 milliliter in volume, and diffusing a gas into the predetermined reaction site. In another embodiment, the invention includes a method of diffusing a gas into a predetermined reaction site no greater than 1 milliliter in volume.

In another aspect, the invention is directed to a method of making a chip and/or a reactor system, e.g., as described in any of the embodiments herein. In yet another aspect, the invention is directed to a method of using a chip and/or a reactor system, e.g., as described in any of the embodiments described herein.

Other advantages and novel features of the invention will become apparent from the following detailed description of various non-limiting embodiments of the invention when considered in conjunction with the accompanying drawings, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

Non-limiting embodiments of the present invention will be described by way of example with reference to the accompanying drawings in which:

FIG. 1 is a graph illustrating oxygen permeability for an embodiment of the invention as used in a bacterial culture;

FIG. 2 is a graph illustrating oxygen permeability for an embodiment of the invention as used in a mammalian cell culture;

FIG. 3 is a cross sectional view of one embodiment of the present invention;

FIG. 4 is a cross sectional view of another embodiment of the present invention.

FIG. 5 is a plot of oxygen transmission versus water vapor transmission for various membranes, including certain membranes used in the invention;

FIG. 6 is an illustration of the dependence of oxygen permeance on film thickness in one embodiment of the invention; and

FIGS. 7A-7D illustrates certain membranes of the invention in fluid communication with various reaction sites.

### DETAILED DESCRIPTION

The present invention is directed to materials and reactor systems having humidity and/or gas control. The material may have high oxygen permeability and/or low water vapor permeability. In some cases, the material may have sufficient permeance and/or permeability to allow cell culture to occur in a chip or other reactor system using the material. In certain embodiments, the material may be positioned adjacent to or abut a reaction site within a chip or reactor; in other embodiments, the material may be positioned such that it is in fluidic communication with the reaction site. The material may also be porous and/or transparent in some cases. In one set of embodiments, the material include a polymer that is branched, and/or contains bulky side groups that allow the polymer to have a more open structure. In some cases, the material may include two or more layers. Each layer may have a desired property, which may include, for example, permeability, transparency, cytophilicity, biophilicity, hydrophilicity, or a structural feature. In some embodiments, the material may be chosen so as to promote cell growth within the chip or reactor.

The following applications are incorporated herein by reference in their entirety: International Patent Publication No. WO 01/68257, published Sep. 20, 2001, entitled “Microreactor,” by Jury, et al.; U.S. Patent Publication No. 2003/0077817, published Apr. 24, 2003, entitled “Microfermentor Device and Cell Based Screening Method,” by A. Zarur, et al.; U.S. Provisional Patent Application Serial No. 60/386,323, filed Jun. 5, 2002, entitled “Materials and Reactors having Humidity and Gas Control,” by Rodgers, et al.; U.S. Provisional Patent Application Serial No. 60/386,322, filed Jun. 5, 2002, entitled “Reactor Having Light-Interacting Component,” by Miller, et al.; a commonly-owned U.S. patent application filed on even date herewith, entitled “Reactor Systems Responsive to Internal Conditions”; a commonly-owned U.S. patent application filed on even date herewith, entitled “Systems and Methods for Control of Reactor Environments,”; a commonly-owned U.S. patent application filed on even date herewith, entitled “Microreactor Systems and Methods,”; a commonly-owned U.S. patent application filed on even date herewith, entitled “System and Method for Process Automation,”; a commonly-owned U.S. patent application Ser. No. filed on even date herewith, entitled “Reactor Systems Having a Light-Interacting Component,”; and a commonly-owned U.S. patent application filed on even date herewith, entitled “Apparatus and Method for Manipulating Substrates”.

A “chip,” as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.). A chip can be connected to or inserted into a larger framework defining an overall reaction system. The system can be defined primarily by other chips, chassis, cartridges, cassettes, and/or by a larger machine or set of conduits or channels, sources of reactants, cells, and/or nutrients, outlets, sensors, actuators, and/or controllers. Typically, the chip can be a generally flat or planar article (i.e., having one dimension that is relatively small compared to the other dimensions); however, in some cases, the chip can be a non-planar article, for example, the chip may have a cubical shape, a solid or block shape, etc.

As used herein, a “membrane” is a three-dimensional material having any shape such that one of the dimensions is substantially smaller than the other dimensions. In some cases, the membrane may be generally flexible or non-rigid. As an example, a membrane may be a rectangular or circular material with a length and width on the order of millimeters, centimeters, or more, and a thickness of less than a millimeter, and in some cases, less than 100 microns, less than 10 microns, or less than 1 micron. Some membranes are semipermeable membranes, which those of ordinary skill in the art will recognize to be membranes permeable with respect to at least one species, but not readily permeable with respect to at least one other species. For example, a semipermeable membrane may allow oxygen to permeate across it, but not allow water vapor to do so, or allows water vapor to permeate it, but at a permeability that is at least an order of magnitude less. Or a semipermeable membrane may be selected to allow water to permeate across it, but not certain ions. Some membranes are transparent to particular light (e.g. infrared, UV, or visible light; light of a wavelength with which a device utilizing the membrane interacts; visible light if not otherwise indicted). Where a membrane is “substantially transparent,” it absorbs no more than 50% of light, or in other embodiments no more than 25% or 10% of light, as described more fully herein. In some cases, a membrane may be both semipermeable and substantially transparent.

As used herein, a “reactor” is the combination of components including a reaction site, any chambers (including reaction chambers and ancillary chambers), channels, ports, inlets and/or outlets (i.e., leading to or from a reaction site), sensors, actuators, membranes, and the like, which, together, operate to promote and/or monitor a biological, chemical, or biochemical reaction, interaction, operation, or experiment at a reaction site, and which can be part of a chip. Examples of reactors include chemical or biological reactors and cell culturing devices, as well as the reactors described in International Patent Application Serial No. PCT/US01/07679, published on Sep. 20, 2001 as WO 01/68257, incorporated herein by reference. Reactors can include one or more reaction sites or chambers. The reactor may be used for any chemical, biochemical, and/or biological purpose, for example, cell growth, pharmaceutical production, chemical synthesis, hazardous chemical production, drug screening, materials screening, drug development, chemical remediation of warfare reagents, or the like. In one set of embodiments, a reactor of the invention comprises a matrix or substrate of a few millimeters to centimeters in size, containing channels with dimensions on the order of, e.g., tens or hundreds of micrometers. Reagents of interest may be allowed to flow through these channels, for example to a reaction site, or between different reaction sites, and the reagents may be mixed or reacted in some fashion. The products of such reactions can be recovered, separated, and treated within the system in certain cases.

As used herein, a “reaction site” is defined as a site within a reactor that is constructed and arranged to produce a physical, chemical, biochemical, and/or biological reaction during use of the reactor. More than one reaction site may be present within a reactor or a chip in some cases, for example, at least 5 reaction sites, at least 7 reaction sites, at least 10 reaction sites, at least 20 reaction sites, at least 30 reaction sites, or at least 50 reaction sites or more may be present within a reactor or a chip. The reaction site may be defined as a region where a reaction is allowed to occur; for example, the reactor may be constructed and arranged to cause a reaction within a channel, one or more chambers, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells. Thus, in some cases, the reaction site may be defined as a region surrounding a location where cells are to be placed within the reactor, for example, a cytophilic region within the reactor.

Many embodiments and arrangements of the invention are described with reference to a chip, or to a reactor, and those of ordinary skill in the art will recognize that the invention can apply to either or both. For example, a channel arrangement may be described in the context of one, but it will be recognized that the arrangement can apply in the context of the other (or, typically, both: a reactor which is part of a chip). It is to be understood that all descriptions herein that are given in the context of a reactor or chip apply to the other, unless inconsistent with the description of the arrangement in the context of the definitions of “chip” and “reactor” herein.

In some embodiments, the reaction site may be defined by geometrical considerations. For example, the reaction site may be defined as a chamber in a reactor, a channel, an intersection of two or more channels, or other location defined in some fashion (e.g., formed or etched within a substrate that can define a reactor and/or chip). Other methods of defining a reaction site are also possible. In some embodiments, the reaction site may be artificially created, for example, by the intersection or union of two or more fluids (e.g., within one or several channels), or by constraining a fluid on a surface, for example, using bumps or ridges on the surface to constrain fluid flow. In other embodiments, the reaction site may be defined through electrical, magnetic, and/or optical systems. For example, a reaction site may be defined as the intersection between a beam of light and a fluid channel.

The volume of the reaction site can be very small in certain embodiments. Specifically, the reaction site may have a volume of less than one liter, less than about 100 ml, les than about 10 ml, less than about 5 ml, less than about 3 ml, less than about 2 μl, less than about 1 ml, less than about 300 microliters, less than about 100 microliters, less than about 30 microliters, or less than about 10 microliters in various embodiments. The reaction site may also have a volume of less than about 5 microliters, or less than about 1 microliter in certain cases.

In some cases, cells can be present at the reaction site, and sensor(s) associated with the chip or reactor may be able to determine the number of cells, the density of cells, the status or health of the cell, the cell type, the physiology of the cells, etc. In certain cases, the reactor can also maintain or control one or more environmental factors associated with the reaction site, for example, in such a way as to support a chemical reaction or a living cell. In one set of embodiments, a sensor may be connected to an actuator and/or a microprocessor able to produce an appropriate change in an environmental factor within the reaction site. The actuator may be connected to an external pump, the actuator may cause the release of a substance from a reservoir, or the actuator may produce sonic or electromagnetic energy to heat the reaction site or selectively kill a type of cell susceptible to that energy. The reactor can include one or more than one reaction site, and one or more than one sensor, actuator, processor, and/or control system associated with the reaction site(s). It is to be understood that any reaction site or a sensor technique disclosed herein can be provided in combination with any combination of other reaction sites and sensors.

As used herein, a “channel” is a conduit associated with a reactor (within, leading to, or leading from a reaction site) that is able to transport one or more fluids specifically from one location to another, for example, from an inlet of the reactor to a reaction site, as further described below. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. Of course, the number of channels, the shape or geometry of the channels, and the placement of channels within the chip can be determined by those of ordinary skill in the art.

Chips of the invention may also include a plurality of inlets and/or outlets that can receive and/or output any of a variety of reactants, products, and/or fluids, for example, directed towards one or more reactors and/or reaction sites. At least a portion of the plurality of inlets and/or outlets may be in fluid communication with one or more reaction sites within the chip. In some cases, the inlets and/or outlets may also contain one or more sensors and/or actuators, as further described below. Essentially, the chip may have any number of inlets and/or outlets from one to tens of hundreds that can be in fluid communication with one or more reactors and/or reaction sites. Less than 5 or 10 inlets and/or outlets may be provided to the reactor and/or reaction site(s) for certain reactions, such as biological or biochemical reactions. In some cases each reactor may have around 25 inlets and/or outlets, in other cases around 50 inlets and/or outlets, in still other cases around 75 inlets and/or outlets, or around 100 or more inlets and/or outlets in still other cases.

As one example, the inlets and/or outlets of the chip, directed to one or more reactors and/or reaction sites may include inlets and/or outlets for a fluid such as a gas or a liquid, for example, for a waste stream, a reactant stream, a product stream, an inert stream, etc. In some cases, the chip may be constructed and arranged such that fluids entering or leaving reactors and/or reaction sites do not substantially disturb reactions that may be occurring therein. For example, fluids may enter and/or leave a reaction site without affecting the rate of reaction in a chemical, biochemical, and/or biological reaction occurring within the reaction site, or without disturbing and/or disrupting cells that may be present within the reaction site. Examples of inlet and/or outlet gases may include, but are not limited to, CO₂, CO, oxygen, hydrogen, NO, NO₂, water vapor, nitrogen, ammonia, acetic acid, etc. As another example, an inlet and/or outlet fluid may include liquids and/or other substances contained therein, for example, water, saline, cells, cell culture medium, blood or other bodily fluids, antibodies, pH buffers, solvents, hormones, carbohydrates, nutrients, growth factors, antifoaming agents (e.g., to prevent production of foam and bubbles), proteins, antibodies, and the like. The inlet and/or outlet fluid may also include a metabolite in some cases. A “metabolite,” as used herein, is any molecule that can be metabolized by a cell. For example, a metabolite may be or include an energy source such as a carbohydrate or a sugar, for example, glucose, fructose, galactose, starch, corn syrup, and the like. Other example metabolites include hormones, enzymes, proteins, signaling peptides, amino acids, etc.

The inlets and/or outlets may be formed within the chip by any suitable technique known to those of ordinary skill in the art, for example, by holes or apertures that are punched, drilled, molded, milled, etc. within the chip or within a portion of the chip, such as a substrate layer. In some cases, the inlets and/or outlets may be lined, for example, with an elastomeric material. In certain embodiments, the inlets and/or outlets may be constructed using self-sealing materials that may be re-usable in some cases. For example, an inlet and/or outlet may be constructed out of a material that allows the inlet and/or outlet to be liquid-tight (i.e., the inlet and/or outlet will not allow a liquid to pass therethrough without the application of an external driving force, but may admit the insertion of a needle or other mechanical device able to penetrate the material under certain conditions). In some cases, upon removal of the needle or other mechanical device, the material may be able to regain its liquid-tight properties (i.e., a “self-sealing” material). Non-limiting examples of self-sealing materials suitable for use with the invention include, for example, polymers such as polydimethylsiloxane (“PDMS”), natural rubber, HDPE, or silicone materials such as Formulations RTV 108, RTV 615, or RTV 118 (General Electric, New York, N.Y.).

In some embodiments, the chip of the present invention may include very small elements, for example, sub-millimeter or microfluidic elements. For example, in some embodiments, the chip may include at least one reaction site having a cross sectional dimension of no greater than, for example, 100 mm, 80 mm, 50 mm, or 10 mm. In some embodiments, the reaction site may have a maximum cross section no greater than, for example, 100 mm, 80 mm, 50 mm, or 10 mm. As used herein, the “cross section” refers to a distance measured between two opposed boundaries of the reaction site, and the “maximum cross section” refers to the largest distance between two opposed boundaries that may be measured. In other embodiments, a cross section or a maximum cross section of a reaction site may be less than 5 mm, less than 2 mm, less than 1 mm, less than 500 micrometers, less than 300 micrometers, less than 100 micrometers, less than 10 micrometers, or less than 1 micrometer or smaller. As used herein; a “microfluidic chip” is a chip comprising at least one fluidic element having a sub-millimeter cross section, i.e., having a cross section that is less than 1 mm. As one particular non-limiting example, a reaction site may have a generally rectangular shape, with a length of 80 mm, a width of 10 mm, and a depth of 5 mm.

While one reaction site may be able to hold and/or react a small volume of fluid as described herein, the technology associated with the invention also allows for scalability and parallelization. With regard to throughput, an array of many reactors and/or reaction sites within a chip, or within a plurality of chips, can be built in parallel to generate larger capacities. Additionally, an advantage may be obtained by maintaining production capacity at the small scale of reactions typically performed in the laboratory, with scale-up via parallelization. It is a feature of the invention that many reaction sites may be arranged in parallel within a reactor of a chip and/or within a plurality of chips. Specifically, at least five reaction sites can be constructed to operate in parallel, or in other cases at least about 7, about 10, about 50, about 100, about 500, about 1,000, about 5,000, about 10,000, about 50,000, or even about 100,000 or more reaction sites can be constructed to operate in parallel. In some cases, the number of reaction sites may be selected so as to produce a certain quantity of a species or product, or so as to be able to process a certain amount of reactant. Of course, the exact locations and arrangement of the reaction site(s) within the reactor or chip will be a function of the specific application.

Additionally, any embodiment described herein can be used in conjunction with a collection chamber connectable ultimately to an outlet of one or more reactors and/or reaction sites of a chip. The collection chamber may have a volume of greater than 10 milliliters or 100 milliliters in some cases. The collection chamber, in other cases, may have a volume of greater than 100 liters or 500 liters, or greater than 1 liter, 2 liters, 5 liters, or 10 liters. Large volumes may be appropriate where the reactors and/or reaction sites are arranged in parallel within one or more chips, e.g., a plurality of reactors and/or reaction sites may be able to deliver a product to a collection chamber.

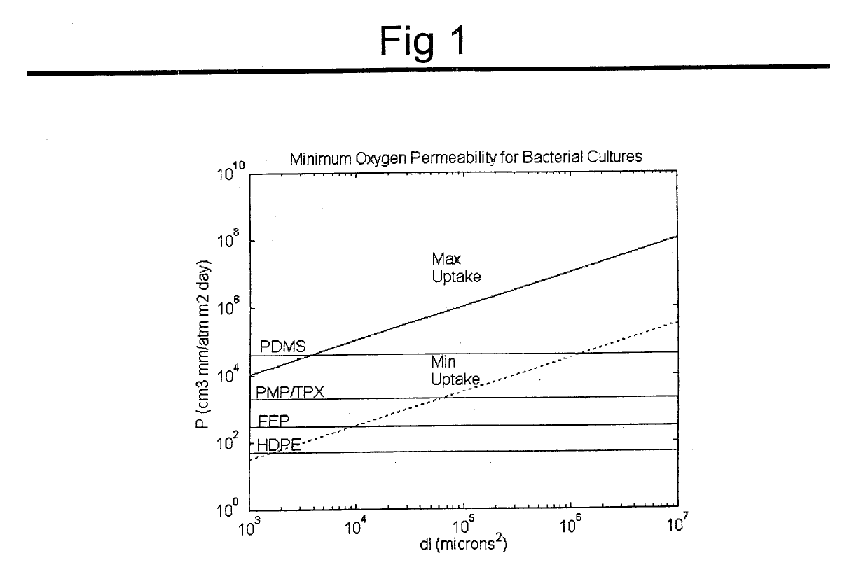

In some embodiments, the reaction site(s) and/or the channels in fluidic communication with the reaction site(s) are free of active mixing elements. In these embodiments, the reactor of the chip can be constructed in such a way as to cause turbulence in the fluids provided through the inlets and/or outlets, thereby mixing and/or delivering a mixture of the fluids, preferably without active mixing, where mixing is desired. Specifically, the reactor and/or reaction site(s) may include a plurality of obstructions in the flow path of the fluid that causes fluid flowing through the flow path to mix, for example, as shown in mixing unit 12 in FIG. 1. These obstructions can be of essentially any geometrical arrangement for example, a series of pillars. As used herein, “active mixing elements” is meant to define mixing elements such as blades, stirrers, or the like, which are movable relative to the reaction site(s) and/or channels themselves, that is, movable relative to portion(s) of the reactor defining a reaction site a or a channel.

The term “determining,” as used herein, generally refers to the measurement and/or analysis of a substance (e.g., within a reaction site), for example, quantitatively or qualitatively, or the detection of the presence or absence of the substance. “Determining” may also refer to the measurement and/or analysis of an interaction between two or more substances, for example, quantitatively or qualitatively, or by detecting the presence or absence of the interaction. Examples of techniques suitable for use in the invention include, but are not limited to, gravimetric analysis, calorimetry, pressure or temperature measurement, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements, including nephelometry.

Chips of the invention can be constructed and arranged such that they are able to be stacked in a predetermined, prealigned relationship with other, similar chips, such that the chips are all oriented in a predetermined way (e.g., all oriented in the same way) when stacked together. When a chip of the invention is designed to be stacked with other, similar chips, it often can be constructed and arranged such that at least a portion of the chip, such as a reaction site, is in fluidic communication with one or more of the other chips and/or reaction sites within other chips. This arrangement can find use in parallelization of chips, as discussed herein.

In one set of embodiments, the chip is constructed and arranged such that the chip is able to be stably connected to a microplate. As used herein, “stably connected” refers to systems in which two components are connected such that a specific motion or force is necessary to disconnect the two components from each other, i.e., the two components cannot be dislodged by random vibration or displacement (e.g., accidentally lightly bumping a component). The components can be stably connected by way of pegs, screws, snap-fit components, matching sets of indentations and protrusions, or the like. A “microplate” is also sometimes referred to as a “microtiter” plate, a “microwell” plate, or other similar terms known to the art. The microplate may include any number of wells. For example, as is typically used commercially, the microplate may be a six-well microplate, a 24-well microplate, a 96-well microplate, a 384-well microplate, or a 1,536-well microplate. The wells may be of any suitable shape, for example, cylindrical or rectangular. The microplate may also have other numbers of wells and/or other well geometries or configurations, for instance, in certain specialized applications.

In another set of embodiments, one or more reaction sites may be positioned in association with a chip such that, when the chip is stably connected to other chips and/or microplates, one or more reaction sites of the chip are positioned or aligned to be in chemical, biological, or biochemical communication with, or chemically, biologically, or biochemically connectable with one or more reaction sites of the other chip(s) and/or one or more wells of the microplate(s). “Alignment,” in this context, can mean complete alignment, such that the entire area of the side of a reaction site adjacent another reaction site or well completely overlaps the other reaction site or well, and vice versa, or at least a portion of the reaction site can overlap at least a portion of an adjacent reaction site or well. “Chemically, biologically, or biochemically connectable” means that the reaction site is in fluid communication with another reaction site or well (i.e., fluid is free to flow from one to the other); or is fluidly connectable to the other site or well (e.g., the two are separated from each other by a wall or other component that can be punctured or ruptured, or a valve in a conduit connecting the two can be opened); or the reaction site and other site or well are arranged such that at least some chemical, biological, or biochemical species can migrate from one to the other, e.g., across a semipermeable membrane. As examples, a chip may have six reaction sites that are constructed and arranged to be aligned with the six wells of a 6-well microplate when the chip is stably connected with the microplate (e.g., positioned on top of the microplate), a chip having 96 reaction sites may be constructed and arranged such that the 96 wells are constructed and arranged to be aligned with the 96 wells of a 96-well microplate when the chip is stably connected with the microplate, etc. Of course, in some cases, the chip may be constructed and arranged such that a single reaction site of the chip is aligned with more than one microplate well and/or more than one other reaction site, and/or such that more than one microplate well and/or more than one other reaction site is aligned with a single reaction site of the chip.

Chips of the invention also may be constructed and arranged such that at least one reaction site and/or reactor of the chip is in fluid communication with, and/or chemically, biologically, or biochemically connectable to an apparatus constructed and arranged to address at least one well of a microplate, for example, an apparatus that can add species to and/or remove species from wells of microplates, and/or can test species within wells of a microplate. In this arrangement, the apparatus may add and/or remove species to/from a reaction site of a chip, and/or test species at reaction sites. In this embodiment, the reaction sites typically are arranged in alignment with wells of the microplate.

Chips of the invention can be substantially liquid-tight in one set of embodiments. As used herein, a “substantially liquid-tight chip” or a “substantially liquid-tight reactor” is a chip or reactor, respectively, that is constructed and arranged, such that, when the chip or reactor is filled with a liquid such as water, the liquid is able to enter or leave the chip or reactor solely through defined inlets and/or outlets of the chip or reactor, regardless of the orientation of the chip or reactor, when the chip is assembled for use. In this set of embodiments, the chip is constructed and arranged such that when the chip or reactor is filled with water and the inlets and/or outlets sealed, the chip or reactor has an evaporation rate of less than about 100 microliters per day, less than about 50 microliters per day, or less than about 20 microliters per day. In certain cases, a chip or reactor will exhibit an unmeasurable, non-zero amount of evaporation of water per day. The substantially liquid-tight chip or reactor can have a zero evaporation rate of water in other cases.

Chips of the invention can be fabricated using any suitable manufacturing technique for producing a chip having one or more reactors, each having one or multiple reaction sites, and the chip can be constructed out of any material or combination of materials able to support a fluidic network necessary to supply and define at least one reaction site. For example, the chip may be fabricated by etching silicon or other substrates, for example, via standard lithographic techniques. The chip may also be fabricated using microassembly or micromachining methods, for example, stereolithography, laser chemical three-dimensional writing methods, modular assembly methods, replica molding techniques, injection molding techniques, milling techniques, and the like as are known by those of ordinary skill in the art. The chip may also be fabricated by patterning multiple layers on a substrate, for example, as further described below, or by using various known rapid prototyping or masking techniques. Examples of materials that can be used to form chips include polymers, glasses, metals, ceramics, inorganic materials, and/or a combination of these. In some cases, the chip may be formed out of a material that can be etched to produce a reactor, reaction site and/or channel. For example, the chip may comprise an inorganic material such as a semiconductor, fused silica, quartz, or a metal. The semiconductor material may be, for example, but not limited to, silicon, silicon nitride, gallium arsenide, indium arsenide, gallium phosphide, indium phosphide, gallium nitride, indium nitride, other Group III/V compounds, Group II/VI compounds, Group III/V compounds, Group IV compounds, and the like, for example, compounds having three or more elements. The semiconductor material may also be formed out of combination of these and/or other semiconductor materials known in the art. In some cases, the semiconductor material may be etched, for example, via known processes such as lithography. In certain embodiments, the semiconductor material may have the from of a wafer, for example, as is commonly produced by the semiconductor industry.

In some embodiments, a chip of the invention may be formed from or include a polymer, such as, but not limited to, polyacrylate, polymethacrylate, polycarbonate, polystyrene, polyethylene, polypropylene, polyvinylchloride, polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The chip may also be formed from composite materials, for example, a composite of a polymer and a semiconductor material.

In some embodiments, the chip, or at least a portion thereof, is rigid, such that the chip is sufficiently sturdy in order to be handled by commercially-available microplate-handling equipment, and/or such that the chip does not become deformed after routine use. Those of ordinary skill in the art are able to select materials or a combination of materials for chip construction that meet this specification, while meeting other specifications for use for which a particular chip is intended.

In certain embodiments, the chip may include a sterilizable material. For example, the chip may be sterilizable in some fashion to kill or otherwise deactivate biological cells (e.g., bacteria), viruses, etc. therein, before the chip is used or re-used. For instance, the chip may be sterilized with chemicals, radiated (for example, with ultraviolet light and/or ionizing radiation), heat-treated, or the like. Appropriate sterilization techniques and protocols are known to those of ordinary skill in the art. For example, in one embodiment, the chip is autoclavable, i.e., the chip is constructed and arranged out of materials able to withstand commonly-used autoclaving conditions (e.g., exposure to temperatures greater than about 100° C. or about 120° C., often at elevated pressures, such as pressures of at least one atmosphere), such that the chip, after sterilization, does not substantially deform or otherwise become unusable. Another example of a sterilization technique is exposure to ozone. In another embodiment, the chip is able to withstand ionizing radiation, for example, short wavelength, high-intensity radiation, such as gamma rays, electron-beams, or X-rays. In some cases, ionizing radiation may be produced from a nuclear reaction, e.g., from the decay of ⁶⁰Co or ¹³⁷CS.

In one set of embodiments, at least a portion of the chip may be fabricated without the use of adhesive materials. For example, at least two components of a chip (e.g., two layers of the chip if the chip is a multi-layered structure, a layer or substrate of the chip and a membrane, two membranes, an article of the chip and a component of a microfluidic system, etc.) may be fastened together without the use of an adhesive material. For example, the components may be connected by using methods such as heat sealing, sonic welding, via application of a pressure-sensitive material, and the like. In one embodiment, the components may be held in place mechanically. For example, screws, posts, cantilevers, etc. may be used to mechanically hold the chip (or a portion thereof) together.

In other embodiments, the two components of the chip may be joined together using techniques such as, but not limited to, heat-sealing methods (e.g., or more components of the chip may be heated to a temperature greater than the glass transition temperature or the melting temperature of the component before being joined to other components), or sonic welding techniques (e.g., vibration energy such as sound energy may be applied to one or more components of the chip, allowing the components to at least partially liquefy or soften).

In another set of embodiments, two or more components of the chip may be joined using an adhesive material. As used herein, an “adhesive material” is given its ordinary meaning as used in the art, i.e., an auxiliary material able to fasten or join two other materials together. Non-limiting examples of adhesive materials suitable for use with the invention include silicone adhesives such as pressure-sensitive silicone adhesives, neoprene-based adhesives, and latex-based adhesives. The adhesive may be applied to one or more components of the chip using any suitable method, for example, by applying the adhesive to a component of the chip as a liquid or as a semi-solid material such as a viscoelastic solid, applying the adhesive on a component using transfer tape, applying a pressure-sensitive adhesive, etc. In another embodiment, the adhesive may be applied to at least a component of the chip using a solvent-bonding system.

In some embodiments of the invention, the chip may be constructed and arranged such that one or more reaction sites can be defined, at least in part, by two or more components fastened together as previously described (i.e., with or without an adhesive). In some cases, a reaction site may be free of any adhesive material adjacent to or otherwise in contact with one or more surfaces defining the reaction site, and this can be advantageous when an adhesive might otherwise leach into fluid at the reaction site. Of course, an adhesive may be used elsewhere in the chip, for example, in other reaction sites. Similarly, in certain cases, a reaction site may be constructed using adhesive materials, such that at least a portion of the adhesive material used to construct the reaction site remains within the chip such that it is adjacent to or otherwise remains in contact with one or more surfaces defining the reaction site. Of course, other components of the chip may be constructed without the use of adhesive materials, as previously discussed.

In one aspect, the present invention is directed to a chip able to control gases or humidity therein. The present invention, in some embodiments, may allow humidity control to be passive and built into a chip that may be used to, for example, conduct chemical or biochemical reactions, or culture cells. In one embodiment, humidity control or maintenance may be provided to the chip in the form of a humidity controller and/or a film, optionally with low water permeability relative to the oxygen permeability. As used herein, a “humidity controller” is a device that allows certain gases, such as oxygen, carbon dioxide, or nitrogen to enter the chip, but inhibits the passage of water vapor into the chip. The humidity controller may allow passage of small amounts of water vapor into the chip, but does not allow as much water vapor to enter the chip as at least one other gas, e.g. those listed above. Examples include, but are not limited to, membranes and thin films (e.g., films having a thickness of less than 2 mm). In some embodiments, the humidity controller may be positioned as, or in, a wall of the chip, such as within a wall of a reactor unit or reaction site. In other embodiments, the humidity controller may be positioned such that it is in fluid communication with one or more reaction sites. In some embodiments, each of the reaction sites in the chip may be adjacent to, and/or in fluid communication with a humidity controller. In some cases, the humidity controller may substantially seal at least a portion of the chip.

Humidity controllers of the invention can be made of a humidity control material designed to maximize gas and/or minimize water vapor passage therethrough. The humidity control material of the present invention may allow the passage of certain desired gases, such as oxygen and/or carbon dioxide, while inhibiting the passage of other gases, for example, water vapor. The material of the present invention is suitable for use as a humidity controller in a chip, but is not limited to such uses; rather, the material could be used anywhere where water vapor or other specified gases are to be kept in or out, while allowing the passage of oxygen and/or other gases. For example, the humidity control material of the present invention may be useful in greenhouses or wound dressings.

In one set of embodiments, the humidity control material may include a membrane or a thin film selected to control the passage of gases and/or water vapor therethrough. In one embodiment, the humidity controller is a membrane or a thin film having a desired permeability to one or more gases. The membrane or thin film may be positioned anywhere in the chip where it is able to affect one or more reaction sites in some fashion. For example, the membrane or thin film may be positioned such that it defines the surface of one or more reaction sites.

In one set of embodiments, the membrane or thin film has a thickness of greater than about 10 micrometers, in some cases greater than about 25 micrometers, in some cases greater than about 50 micrometers, in some cases greater than about 75 micrometers, in some cases greater than about 100 micrometers, or in some cases greater than about 150 micrometers while still allowing sufficient oxygen transport therethrough, for instance, to enable cell culture to occur, as further described herein. In some cases, a membrane or a thin film having a thickness of greater than about 50 micrometers may be particularly useful, for example, during manufacturing of the chip. The membrane may have a thickness of less than 1 or 2 millimeters in some cases.

In some cases, it may be desired to incorporate the humidity control material into a structural aspect of the chip, or to incorporate structural aspects of the chip into the humidity control material. Where the humidity control material is intended to provide or supplement support, or will not itself be otherwise adequately supported, the humidity control material may also include a support layer. A support layer may comprise any material or materials that provides desired support. For example, the support layer may include one of the layers that may otherwise be included in the humidity control material for permeability, such as polydimethylsiloxane or polyfluoroorganic materials, or the support layer may comprise a different material, such as glass (for example, PYREX® glass by Corning Glass of Corning, N.Y.; or indium/tin-coated glass), latex, silicon, or the like. The support layer may be positioned anywhere within the humidity control material, for example, as an outer layer or an intermediate layer, and may be positioned to help protect one or more delicate layers. In some embodiments of the present invention, the use of a support layer may allow a large portion, or nearly all of a reaction site, reactor, or chip to be constructed of the humidity control material. Preferably, the support layer does not significantly impact the permeability of the humidity control material, or the change in permeability may be accounted for in the design of the humidity control material.

Where the chip of the present invention is intended for use with materials, such as reactants, that may damage, reduce the function, or otherwise react with or cause the humidity control material to deteriorate, the membrane may include a protection layer. The protection layer may be positioned as any component of the humidity control material, for example, as a surface layer, or interposed between a sensitive portion of the humidity control material and the material or environment that may adversely affect it. For example, the protection layer may be positioned on an inner surface of the humidity control material, particularly where the harmful material is within the chip, or on the outer surface of the humidity control material, particularly where the harmful material is outside the chip. The protection layer may also be positioned between other layers, so long as it is able to perform is protective function. Preferably, the protection layer does not significantly impact the permeability of the humidity control material, or the change in permeability may be accounted for in the design of the humidity control material.

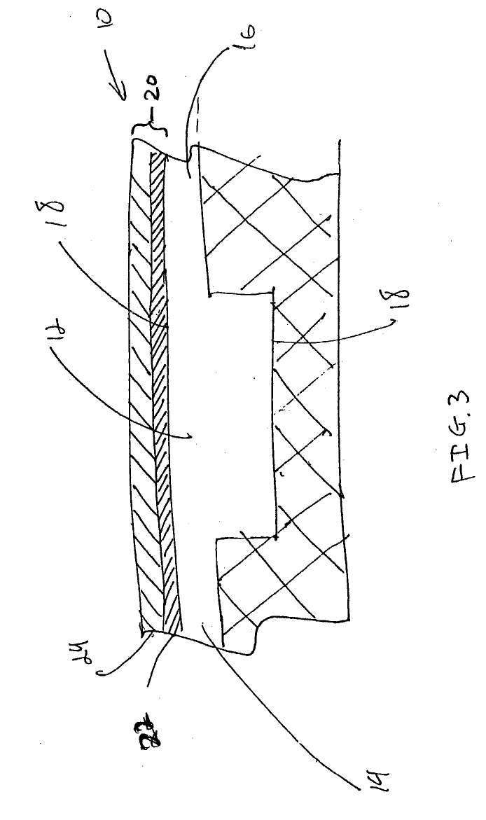

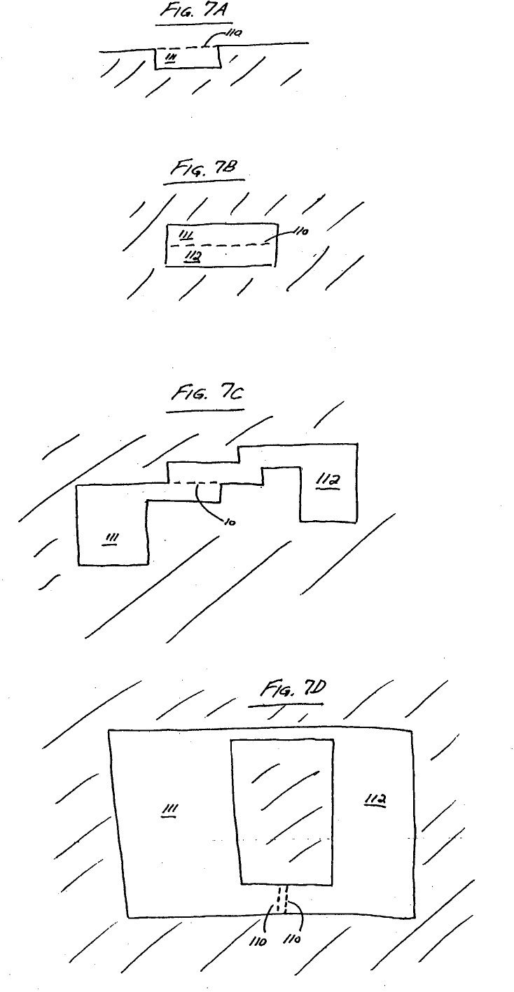

As an example, a chip 10 including a humidity controller according to one embodiment of the present invention is illustrated in FIG. 3. This chip includes a reaction site 12, an inlet 14, an outlet 16, and an inner wall 18. Inner wall 18 is defined on one side by a humidity controller 20. Humidity controller 20, in this embodiment, includes a membrane having a first layer 22 and a second layer 24. As examples of other arrangements including a humidity controller, with reference to FIG. 7A, membrane 110, which is a humidity controller, defines a surface of reaction site 111. In FIG. 7B, membrane 110 defines the surface of reaction site 111 and a surface of reaction site 112. As another example, the membrane can be positioned such that it is in fluidic communication with one or more reaction sites of the chip. In some cases, the membrane may be positioned such that a pathway fluidly connecting a first reaction site with a second reaction site crosses the membrane. For example, in FIGS. 7C and 7D, membrane 110 does not define surfaces of reaction sites 111 or 112, but is positioned such that at least one pathway fluidly connecting reaction site 111 with reaction site 112 crosses membrane 110.

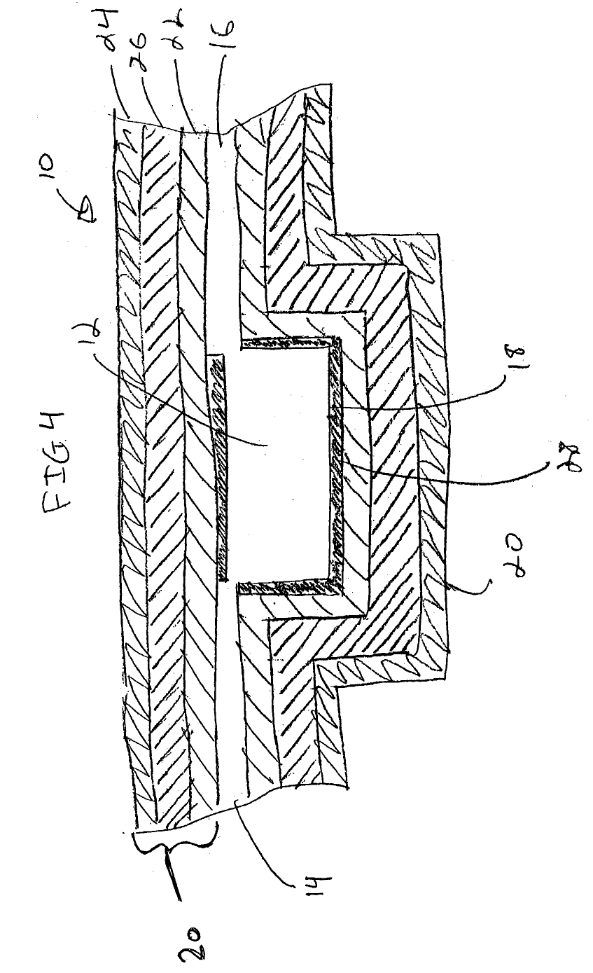

Another embodiment of a chip 10 including a humidity controller is illustrated in FIG. 4. In this embodiment, the humidity controller 20 includes a multi-layer membrane that defines a wall of a reaction chamber 12, and also defines a wall of an inlet and of an outlet. In addition to first and second layers 22 and 24, which are provided primarily for purposes of providing a desired permeability, this membrane also includes a support layer 26 positioned between first and second layers 22, 24. Other arrangements for the permeability-controlling layer(s) and support layer(s) are possible. Also provided in chip 10 is a cell adhesion layer 28 positioned on inner wall 18 of reaction site 12, encouraging cell growth there and not in inlet 14 and outlet 16. In other embodiments, the cell adhesion layer could extend over more, or all, of the surface of humidity controller 20. It should also be appreciated that the geometry of chip 10 as illustrated in FIGS. 3 and 4 is shown by way of illustration only and that many other arrangements and chip geometry may be useful in particular embodiments.

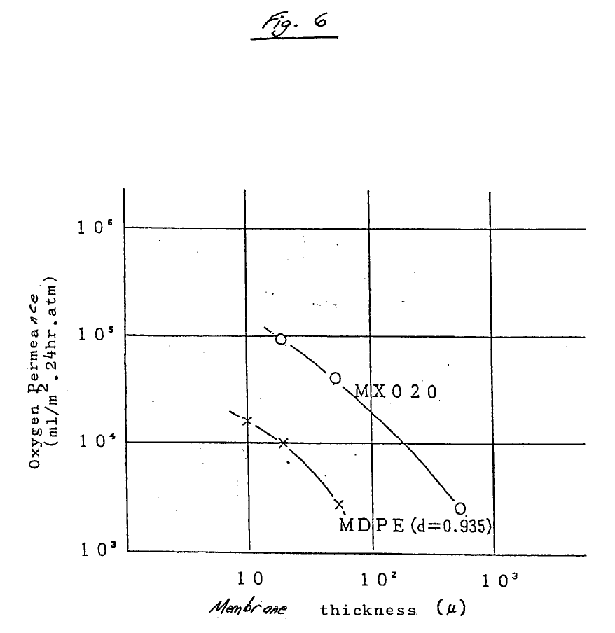

In one set of embodiments, the humidity control material is selected to have a certain permeability and/or a certain permeance. As used herein, the “perineability” of a material is given its ordinary meaning as used in the art, i.e., an intrinsic property that generally describes the ability of a gas to pass through the material. In contrast, as used herein, the “permeance” of a material is the actual rate of gas transport through a sample of a material, i.e., an extrinsic property. The permeance of a sample of material is affected by factors such as the area or thickness of the material, the pressure differential across the material, etc. For example, in FIG. 6, the oxygen permeance of two membranes is shown to be dependent on the membrane's thickness.

A chip of the present invention, in one set of embodiments, may include a humidity control material (e.g., a membrane or a thin film) having a permeability to oxygen greater than about 3.9×10⁻⁸ cm³/s, and in some cases greater than about 4.3×1 0⁻⁸ cm³/s, and/or a permeability to water vapor lower than about 1.7×10⁻⁷ cm³/s, and in some cases lower than about 1.0×10⁻⁷ cm³/s. It should be appreciated that, while control of oxygen is used as an example herein, other gases such as nitrogen or carbon dioxide may be controlled instead, at permeabilities as noted above, or a combination of gases may be controlled. It should also be appreciated that while, in the example of cells further described below, the lower limit of oxygen transfer and the upper limit of water vapor transfer may typically be desired to be controlled, in other applications, for example, in a chemical synthesis operation, it may be desired to control other parameters, for example, the upper limit of oxygen transfer and lower limit of water vapor transfer, or the lower and upper limits of other gases such as nitrogen or carbon dioxide.

The humidity control material of the present invention may be used in a wide variety of reactions and interactions. One example of a reaction is cell culture, for example to maintain a cell culture, to increase the number of available cells or cell types, or to produce a desirable cellular product. In some cases, the humidity control material may allow sufficient oxygen to enter by diffusion therethrough to support cell growth. In certain cases, the humidity control material may also be largely impermeable to microorganisms and other cells, for example to prevent contamination. Preferably, the material has low toxicity.

In embodiments where the invention is used in connection with culturing cells, cell culturing may take place over varying lengths of time, depending on the cells being cultured and other factors known to those of ordinary skill in the art. Thus, the design of the chip and the nature of the humidity control material may be adapted to the culture time. For example, the chip or humidity control material may be designed to allow it to withstand the time needed for the culture and is preferably designed to be able to be reused many times. In various embodiments, cell cultures may be performed in 24 hours, 48 hours, 1 week, 2 weeks, 4 weeks, 6 weeks, 3 months, 1 year, continuously, or any other time required for a specific cell culture.

In some cases, the humidity control material is selected to have a permeability and/or a permeance to one or more gases that corresponds to a range acceptable for culturing certain cells. For example, the humidity control material may have a permeability and/or permeance to oxygen high enough, and/or a permeability and/or permeance to water vapor low enough, to allow cell culturing. Examples of such permeabilities include the above-described permeabilities. Those of skill in the art will be able to identify specific ranges of permeabilities of certain materials appropriate for successfully culturing particular cells and cell lines, as well as larger cellular groups, such as microbial and mammalian cells, tissues, tissue engineering constructs, etc.

Thus, in one embodiment, the invention includes a method of identifying an oxygen requirement and a humidity requirement of certain cells, selecting a material having an oxygen permeability high enough to meet the oxygen requirement of the cells and a water vapor permeability low enough to meet the humidity requirement of the cells, and culturing the cells in a chip comprising a reaction site. The reaction site has at least a portion thereof formed of the selected material.

Examples of permeability ranges of a humidity control material for use in the invention, for example for use in culturing a broad range of cells, include a permeability to oxygen greater than about 100 (cm³_{STP} mm/m² atm day), and a permeability to water vapor less than about 6×10⁻⁶ (cm³_{STP} mm/m² atm day). As used herein, “STP” refers to “standard temperature and pressure,” referring to a temperature of 273.15K (0° C.) and a pressure of about 10⁵ Pa (1 atm). In another embodiment, the humidity control material may have a permeability to water that is less than about 100 (cm³_{STP} mm/m² atm day) and, in other embodiments, less than about 30 (cm³_{STP} mm/m² atm day) or less than about 10 (cm³_{STP} mm/m² atm day), and an oxygen permeability of at least about 6×10⁶ (cm³_{STP} mm/m² atm day), and in some embodiments, at least about 1×10⁷ (cm³_{STP} mm/m² atm day), and in other embodiments greater than about 3×10⁷ (cm³_{STP} mm/m² atm day) or 1×10⁸ (cm³_{STP} mm/m² atm day). Any combination of oxygen permeability and water vapor permeability listed herein can be used. For microbial cells, an example of a suitable range of oxygen permeability is provided by a membrane having a permeability to oxygen permeability greater than about 1×10³ (cm³_{STP} min/m² atm day) and/or a permeability to water vapor is less than about 6×10⁶ (cm³_{STP} mm/m² atm day). For mammalian cells, an example suitable range is provided by a membrane of the invention having a permeability to oxygen greater than about 100 (cm³_{STP} mm/m² atm day) and a permeability to water vapor lower than about 1×10⁵ (cm³_{STP} mm/m² atm day).

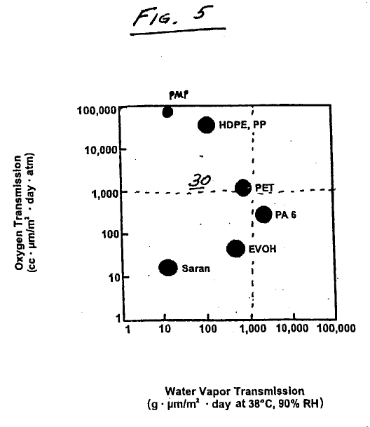

For humidity control materials having a permeability to oxygen and water vapor, in certain cases, it is desired that the material have very high oxygen permeability and very low permeability to water vapor, e.g., as is indicated in FIG. 5 by “goal” region 30. For example, the material may have an oxygen permeability of greater than about 1000 cm³_{STP} micrometer/m² day atm, in some cases greater than about 10,000 cm³_{STP} micrometer/m² day atm, and in some cases greater than about 100,000 cm³_{STP} micrometer/m² day atm, and/or a permeability to water vapor less than about 1000 g micrometer/m² day, in some cases less than about 100 g micrometer/m² day, and in some cases less than about 10 g micrometer/m² day. For instance, as illustrated in FIG. 5, the results of materials such as high density polyethylene (“HDPE”), polyethylene terephthalate (“PET”), polypropylene (“PP”), or poly(4-methylpentene-1) (“PMP”) are shown, and these may be suitable for use with the invention, as further described below. Other materials and combinations of materials are also contemplated, e.g., as further described below.

In some embodiments, the humidity control material does not promote cell adhesion, but may include a cell adhesion layer (or a cell adhesion layer can be provided on the material) that may be any of a wide variety of hydrophilic, cytophilic, and/or biophilic materials. Examples of materials that may be suitable for a cell adhesion layer on a humidity control material include, but are not limited to, polyfluoroorganic materials, polyester, PDMS, polycarbonate, polystyrene, and aluminum oxide. As another example, the humidity control material may include a layer coated with a material that promotes cell adhesion, for example, using an RGD peptide sequence. In some embodiments, it may be desired to modify the surface of a cell adhesion layer, for example, by attachment, binding, soaking or other treatments. Example molecules that promote cell adhesion include, but are not limited to, fibronectin, laminin, albumin or collagen. Where the material includes a cell adhesion layer, the cell adhesion layer may be positioned as an inner layer or a surface layer of the membrane, or may abut an interior of the chip. Preferably, the cell adhesion layer does not significantly impact the permeability or permeance of the humidity control material, or the change in permeability or permeance may be accounted for in the design of the humidity control material.

Some of the materials used to form the humidity control material, and, in some cases, some of the layers thereof, may be selected based on the gas permeabilities of the materials, for example, as previously described. Those of ordinary skill in the art will know of methods of determining the gas permeability of a material. As one particular example method, a sample of a material having a known exposed area and thickness (e.g., a membrane) may be placed between two chambers, and a gas (or a liquid) may be placed in one chamber. The experimental time it takes for the gas (or liquid) to diffuse across the material to the other chamber and detected in a suitable fashion may then be related to the gas (or liquid) permeability of the material.

In one set of embodiments, the humidity control material may include a polymer (e.g., a single polymer type, a co-polymer, a polymer blend, a polymer derivative, etc.). Examples of polymers that may be used within the humidity control material include, but are not limited to, polyfluoroorganic materials such as polytetrafluoroethylenes (e.g., such as those marketed under the name TEFLON® by DuPont of Wilmington, Del., for example, TEFLON® AF) or certain amorphous fluoropolymers; polystyrenes; PP; silicones such as polydimethylsiloxanes; polysulfones; polycarbonates; acrylics such as polymethyl acrylate and polymethyl methacrylate; polyethylenes such as high-density polyethylenes (“HDPE”), low-density polyethylenes (“LDPE”), linear low-density polyethylenes (“LLDPE”), ultra low-density polyethylenes (“ULDPE”) etc.; PET; polyvinylchlorides (“PVC”); nylons such as that marketed under the name DARTEK® by Dupont; and the like. Another example of a suitable material is a BIOFOIL® polymer membrane, made by VivaScience (Hannover, Germany). In one embodiment, the polymer may be poly(4-methylpentene-1) (“PMP”):

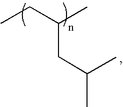

which, in some cases, may have a permeability coefficient for oxygen of about 317.2 (m³_{STP} m/s m Pa). Examples of PMPs include those marketed under the name TPX™ by Mitsui Plastics (White Plains, N.Y.). In other embodiments, the polymer may be poly(4-methylhexene-1), poly(4-methylheptene-1) poly(4-methyloctene-1), etc. In another embodiment, the polymer may be poly(1-trimethlsilyl-1-propyne) (“PTMSP”):

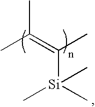

which, in some cases, may have a permeability coefficient for oxygen of about 5.78×10⁵ (cm³_{STP} mm/m² day atm). In some cases, copolymer of these and/or other polymers may be used in the humidity control material.

In some embodiments, the area and thickness of the humidity control material, or a layer or portion thereof, may be used to select a desired degree of permeance and/or permeability. As one example, a more water vapor-permeable material may be made thicker, or its area may be reduced, in order to reduce the amount of water vapor that reaches or leaves the area or region where humidity control is desired. In some cases, the material may be designed such that it is between about 10 micrometers and 2 mm thick. Within this range, the relative thickness of layers within multiple layers or portions of the material may vary. For example, a relatively thick layer of a polyfluoroorganic material and a relatively thin layer of vinylidene chloride may be useful in particular embodiments. As additional examples, a few micrometers of polytetrafluoroethylene may be deposited or coated onto a layer of polydimethylsiloxane, or a few micrometers of HDPE could be co-molded with PDMS.

In some cases, the polymer (or mixture of polymers) used in the humidity control material may be sufficiently hydrophobic such that the polymer is able to retain water (i.e., water vapor is not able to readily transport through the polymer). For instance, the permeability of water through a hydrophobic polymer may be less than about 1000 g micrometer/m² day, 900 g micrometer/m² day, 800 g micrometer/m² day, 600 g micrometer/m² day or less, as previously described.

In certain embodiments, the polymer(s) used in the humidity control material may have a molecular structure open enough to readily allow the transport of oxygen therethrough. For instance, the molecular structure may allow transport of oxygen across the polymer of greater than about 1000 cm³_{STP} micrometer/m² day atm or more, as previously described. In one embodiment, the polymer is sufficiently branched such that the polymer is unable to form a structure under ambient conditions (e.g., a tightly crystalline structure) that limits the transport of oxygen therethrough, for instance, to less than about 1000 cm³_{STP} micrometer/m² day or 500 cm³_{STP} micrometer/m² day.

In another embodiment, the polymer may include a bulky group that prevent the polymer from readily forming a structure under ambient conditions that limits the transport of oxygen therethrough. A “bulky group” on a polymer, as used herein, is a moiety sufficiently large that the polymer is unable to form a crystalline structure under ambient conditions that limits the transport of oxygen therethrough to less than about 1000 cm³_{STP} micrometer/m² day or 500 cm³_{STP} micrometer/m² day. The bulky group may be, for instance, part of the backbone of the polymer or a side chain. Non-limiting examples of bulky side groups include groups containing cyclopentyl moieties, isopropyl moieties, cyclohexyl moieties, phenyl moieties, isobutyl moieties, tert-butyl moieties, cycloheptyl moieties, trimethylsilyl or other trialkylsilyl moieties etc. For example, in one set of embodiments, the polymer may have a structure:

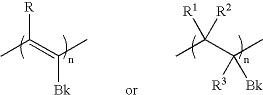

where each R independently comprises at least one atom, and Bk is a bulky group. In some cases, R may be a hydrogen or an alkyl group.

Of course, it should be understood that the polymer may have several or all of the above-described features. For example, the polymer may be a polymer blend or a copolymer that has sufficient hydrophobicity such that the polymer is able to retain water yet have a molecular structure open enough to allow sufficient oxygen permeability therethrough.

In another set of embodiments, the humidity control material of the present invention allows light to pass through it. This may allow the material to be used where light is important, for example, to facilitate a reaction such as a photocatalyzed reaction, to promote cell or plant growth, to cause a biochemical change to occur, or the like. The material may also allow observation of a region, such as a reactor or reaction site, that is protected by the humidity control material, or is located behind a humidity-controlled region. In one embodiment, the humidity control material is translucent, and, in a more preferred embodiment, it is at least substantially transparent. One of skill in the art will recognize that there are varying degrees of translucence and transparence, and will be able to select desired properties based upon a particular application. As used herein, a “substantially transparent” membrane is a membrane that allows electromagnetic radiation to be transmitted through the membrane without significant scattering, such that the intensity of electromagnetic radiation transmitted through the membrane is sufficient to allow the radiation to interact with a substance on the other side of the membrane, such as a chemical, biochemical, or biological reaction, or a cell. In some cases, the membrane is substantially transparent to incident electromagnetic radiation ranging between the infrared and ultraviolet ranges (including visible light) and, in particular, between wavelengths of about 400-410 nm and about 1,000 nm. The substantially transparent membrane may be able to transmit electromagnetic radiation in some cases such that a majority of the radiation incident on the membrane passes through the membrane unaltered, and in some embodiments, at least about 50%, in other embodiments at least about 75%, in other embodiments at least about 80%, in still other embodiments at least about 90%, in still other embodiments at least about 95%, in still other embodiments at least about 97%, and in still other embodiments at least about 99% of the incident radiation is able to pass through the membrane unaltered. In certain cases, the membrane is at least partially transparent to electromagnetic radiation within the above-mentioned wavelength range to the extent necessary to promote and/or monitor a physical, chemical, biochemical, and/or biological reaction occurring within a reaction site, for example as previously described. In other embodiments, the membrane may be transparent to electromagnetic radiation within the above-mentioned wavelength range to the extent necessary to monitor, observe, stimulate and/or control a cell within the reaction site.

In some embodiments, the humidity control material may be porous. For example, the material may have a number-average pore size of greater than about 0.03 micrometers and less than about 2 micrometers. In other embodiments, the pore size of the material may be less than about 1.5 micrometers, less than about 1.0 micrometers, less than about 0.75 micrometers, less than about 0.5 micrometers, less than about 0.3 micrometers, less than about 0.1 micrometers, less than about 0.07 micrometers, and in other embodiments, less than about 0.05 micrometers. In certain cases, the pores are also greater than 0.03 micrometers or greater than 0.08 micrometers.

In certain embodiments, the humidity control material may be formed out of a substance that has a number-average pore size that is also substantially transparent, as previously described. For example, the porous substantially transparent material may include polymers such as polyethylene terephthalate (PET), polysulfone, polycarbonate, acrylics such as polymethyl methacrylate, polyethylene, polypropylene, and the like. In one embodiment, the substantially transparent material is a polyethylene terephthalate membrane having a pore size of 2 micrometers or less, for example, a ROTRAC® capillary membrane made by Oxyphen U.S.A., Inc. (New York, N.Y.).

In some embodiments, the present invention achieves a certain permeability and/or permeance of the humidity control material by combining two or more layers or portions of material. For example, where the humidity control material is a membrane that comprises at least two layers, the layers may be formed out of the same or distinct polymers.

Thus, in one embodiment, the present invention achieves a permeability goal by combining two layers or portions of material. This can be achieved, for example, by including a first, more permeable layer, and a second, less permeable layer; multiple layers may also be used in other embodiments. By combining different materials and adjusting their relative thickness, a desired oxygen and water vapor permeability may be achieved. In one embodiment where the humidity control material comprises two layers or portions, they may be formed out of the same or different materials polymers. For example, the humidity control material may include a first layer including at least about 55% by weight of a first polymer or co-polymer and a second layer comprising no more than about 45% by weight of the first polymer or co-polymer. As another example, the humidity control material may include a first layer including at least about 60%, about 70%, or about 80% by weight of a first polymer or co-polymer and a second layer comprising no more than about 40%, about 30%, or about 20% by weight of the first polymer or copolymer. In some embodiments, the first polymer may comprise about 100% of the first layer and essentially none of the second layer. In some cases, at least a portion of the first layer may be co-polymerized with the second layer.

Some of the materials used to form the humidity control material, and, in some embodiments, some of the layers thereof, may be selected based on the materials' gas permeabilities. Thus, for example, if the humidity control material is a membrane having two layers, the first layer may be a more permeable layer formed from polyfluoroorganic materials, polystyrenes, PVC, polyvinylidene chlorides, nylons, poly(4-methylpentene-1), etc., where the layer has a permeability to oxygen between about 10³ (cm³_{STP} mm/m² atm day) and 10⁵ (cm³_{STP} mm/m² atm day) and a permeability to water vapor between about 10² (cm³_{STP} mm/m² atm day) and 6×10⁶ (cm³_{STP} mm/m² atm day); the second layer may be chosen to have very high permeability to a gas and/or a degree of mechanical stability formed from PDMS, HDPE, LDPE, LLDPE, a thermoplastic elastomer, etc. Of course, the first and second layers may also each include a mixture of materials in some embodiments. For example, one layer may include at least 50% by weight of one material with the balance comprising one or more other materials. In another embodiment, each layer consists essentially of a single material.

Where the humidity control material of the present invention is constructed as a membrane including two or more layers, the two or more layers may be joined in any manner that provides sufficient strength to the membranes. In some cases, the two or more layers may be sufficiently self-supporting and it may not be necessary to join the layers, meaning a space could be left therebetween if desired. In other embodiments, additional layers may be used to support the membrane. In embodiments where it is desired to join the two or more layers to provide mutual support or otherwise, examples of acceptable means of joining the layers include laminating the layers together, at least partially intermixing the layers, and co-polymerizing the layers together. Where the layers are to be intermixed, the resin that will form each layer may be partially or totally intermixed before the membrane is formed. For example, liquid pre-polymers may be mixed and then a curing agent added, or two partially cured layers can be connected with a curing agent between them, curing the layers together.

The chip can include a variety of components for sensing, actuation, or other activity. For example, the chip may include components such as a membrane, a lens, a light source, a waveguide, a circuit such as an integrated circuit, a reservoir (e.g., for a solution), a micromechanical or a MEMS (“microelectromechanical system”) component, a control system, or the like, for example, as further described below. In some embodiments, at least one, two, three or more components are integrally connected to the chip. In certain embodiments, all of the components are integrally connected to the chip.

Other examples of components suitable for use with the invention include pylon-like obstructions placed in the flow path of a stream to enhance mixing within the chip, reactor and/or reaction site, or heating, separation, and/or dispersion units within the chip, reactor and/or reaction site. For example, if a heating unit is present, the heating unit may be a miniaturized, traditional heat exchanger.

In one set of embodiments, a chip of the invention may include a structure adapted to facilitate the reactions or interactions that are intended to take place therein (e.g., within a reaction site). For example, where a chip is intended to function as one or more bioreactors for cell culturing, the chip may include structure(s) able to improve or promote cell growth. For instance, in some cases, a surface of a reaction site may be a surface able to promote cell binding or adhesion, or the reactor and/or reaction site within the chip may include a structure that includes a cell adhesion layer, which may include any of a wide variety of hydrophilic, cytophilic, and/or biophilic materials. As examples, the surface may be ionized, or coated and/or micropatterned with any of a wide variety of hydrophilic, cytophilic, and/or biophilic materials, for example, materials having exposed carboxylic acid, alcohol, and/or amino groups. Examples of materials that may be suitable for a cell adhesion layer include, but are not limited to, polyfluoroorganic materials, polyester, PDMS, polycarbonate, polystyrene, and aluminum oxide. As another example, the structure may include a layer coated with a material that promotes cell adhesion, for example, an RGD peptide sequence, or the structure may be treated in such a way that it is able to promote cell adhesion, for example, the surface may be treated such that the surface becomes relatively more hydrophilic, cytophilic, and/or biophilic. In some embodiments, it may be desired to modify the surface of a cell adhesion layer, for instance with materials that promote cell adhesion, for example, by attachment, binding, soaking or other treatments. Example materials that promote cell adhesion include, but are not limited to, fibronectin, laminin, albumin or collagen. In other embodiments, for example, where certain types of bacteria or anchorage-independent cells are used, the surface may be formed out of a hydrophobic, cytophobic, and/or biophobic material, or the surface may be treated in some fashion to make it more hydrophobic, cytophobic, and/or biophobic, for example, by using aliphatic hydrocarbons and/or fluorocarbons.

In some embodiments, the chip may include a “light-interacting component,” i.e., a component that interacts with light, for example, by producing light, reacting to light, causing a change in a property of light, directing light, altering light, etc. In general, the term “light-interacting component” does not encompass components that passively transmit light without significant modification, alteration, or redirection, such as air, or a plane of glass or plastic. The term “light-interacting component” also does not encompass components that passively absorb essentially all incident light without a response, such as in an opaque material. Examples of light-interacting component include lenses, filters, optical fiber, waveguides, diffraction gratings, mirrors, prisms, etc.

The light-interacting component may include a waveguide in some cases. The term “waveguide” is given its ordinary meaning in the art and may include optical fibers. A waveguide is generally able to receive light and guide or transmit a portion of that light to a destination not within “line-of-sight” communication (although a waveguide can transmit light to a line-of-sight region), e.g., around bends, corners, and similar obstacles. In some embodiments, a waveguide may include a “core” region of material embedded or surrounded, at least in part, by a second “cladding” material, which may have a lower refractive index than the core region.

The waveguide, or at least a portion of the waveguide, may be fashioned out of any material able to transmit or light to or from a reaction site. In some cases, the waveguide may be formed out of a silicon-based material, for example, glass, ion-implanted glass, quartz, silicon, silicon oxide, silicon nitride, silicon carbide, polysilicon, coated glass, conductive glass, indium-tin-oxide glass and the like. In other cases, the waveguide may comprise other transparent or translucent organic or inorganic materials. For example, in certain cases, the waveguide may comprise a polymer including, but not limited to, polyacrylate, polymethacrylate, polycarbonate, polystyrene, polypropylene, polyethylene, polyimide, polyvinylidene fluoride, an ion-exchanged polymer, and fluorinated derivatives of the above.

In one embodiment, the waveguide or a portion thereof may be surrounded by or coated with a highly reflective material, for example, silver or aluminum. In another embodiment, the waveguide may be fashioned such that it comprises a central material (e.g., a core) having a first index of refraction, and a surrounding material (e.g., a cladding) having a second index of refraction. The cladding may have an index of refraction that is less than the index of refraction of the core. In yet another embodiment, the index of refraction of the core or the cladding may vary over the cross section. As one example, the core may be a graded index optical fiber, where the index of refraction is generally highest near the center of the core.

As one example of a waveguide, both the central and surrounding materials forming the waveguide may each be a glass. As another example, a waveguide may be formed out of a polymer and a silicon-based material, such that the material with the lower index of refraction surrounds the material with the higher index of refraction. As yet another example, the waveguide may be constructed out of a single material surrounded by, for example, air or a portion of the chip having a higher index of refraction than the waveguide, thus resulting in a condition where total internal reflection may occur at the waveguide/air or waveguide/chip interface.

The waveguide may be constructed by any suitable technique known to those of ordinary skill in the art, for example, by milling, grinding, or machining (e.g., by cutting or etching a channel into a chip substrate, then depositing material into the channel, optionally using a sealant). The waveguide may also be formed, for example, by depositing layers of materials during the chip fabrication process. The waveguide may also be constructed by laser etching of materials forming the chip, such as glass or plastic, in such a way as to manipulate/alter the refractive index, relative to the surrounding material. In some cases, the refractive index of the etched/non-etched portion may be controlled so as to create a core-cladding structure.

In some embodiments, the light-interacting component may be, or include, a source of light. The light source may be any light source in optical communication with the reaction site. For example, the light source may be external or ambient light, a coherent or monochromatic beam of light such as created in an LED, or a laser such as a semiconductor laser or a quantum well laser. The light source may be integrally connected with a portion of the chip, for example, in a laser diode fabricated as part of the chip, or the light source may be separate from the chip and not integrally connected with it, but still positioned so as to allow optical communication with the reaction site. The light source may produce a single wavelength or a substantially monochromatic wavelength, or a wide range of wavelengths, as previously described. The source of light, in certain embodiments, may also be generated in a chemical reaction or a biological process, such as a chemical reaction that produces photons, for example, a reaction involving GFP (“green fluorescence protein”) or luciferase, or through fluorescence or phosphorescence. For example, incident electrons, electrical current, friction, heat, chemical or biological reactions may be applied to generate light, for example, within a sample located within a reaction site, or from a reaction center located within the chip in optical communication with the reaction site.

In certain cases, the light-interacting component may include a filter, for example, a low pass filter, a high pass filter, a notch filter, a spatial filter, a wavelength-selecting filter, or the like. The filter may be able to, for example, substantially reduce or eliminate a portion of the incident light. In another embodiment, the filter may be able to reduce noise within the incident light, or increase the signal-to-noise ratio of the incident light. In still another embodiment, the filter may be able to polarize the incident light, for example, linearly or circularly.

In some cases, the light-interacting component may include an optical element in optical communication with the reaction site. As used herein, an “optical element” refers to any device able to alter the pathway of light entering or exiting the optical element, for example, by focusing or collimating light, or causing the light to diverge. In certain embodiments, the optical element may disperse light, for example, as in a diverging lens. In other embodiments, the optical element may be, for example, a beamsplitter, an optical coating (e.g., a dichroic, an antireflective, or a reflective coating), an optical grating, a diffraction grating, or the like.

The optical element may be a lens in certain cases. The lens may be any lens, such as a converging or a diverging lens. The lens may be, for example, a meniscus, a plano-convex lens, a piano-concave lens, a double convex lens, a double concave lens, a Fresnel lens, a spherical lens, an aspheric lens, a binary lens, or the like. The optical element may also be a mirror in some instances, such as a planar mirror, a curved mirror, a parabolic mirror, or the like. In other cases, the optical element may disperse light, for example, a diffraction grating or prism.

In certain cases, a material having a different index of refraction may be used. For example, in embodiments in which light reaches the optical element through a waveguide, the optical element may be a material having a different index of refraction than the waveguide. In some cases, the index of refraction of the optical element will be about the same as or more than the index of refraction of the waveguide.

In some cases, a material having a graded index of refraction (a “GRIN” material) may be used as an optical element. The GRIN material may minimize the amount of divergence inherent in light reaching the GRIN material. For example, a material of uniform thickness can be made to act as a lens by varying its refractive index along a cross section of the element. In one embodiment, the GRIN material may redirect divergent rays of light into a parallel arrangement. In another embodiment, the GRIN material does not necessarily have a uniform thickness, and a combination of the graded index of refraction of the material and the shape of the material may be used to focus or collimate the light.

The light-interacting component, in some cases, may include a component that is able to convert light to electricity, such as a photosensor or photodetector, a photomultiplier, a photocell, a photodiode such as an avalanche photodiode, a photodiode array, a CCD chip or the like. The component may be used, in some cases, to determine the state or condition of a substance within a reaction site, for example, through emission, absorbance, scattering, optical density, polarization measurements, etc. In other cases, the component may be used for imaging purposes. In some instances, the component may be used to produce electricity.

In some embodiments of the invention, a reactor and/or a reaction site within a chip may be constructed and arranged to maintain an environment that promotes the growth of living cells. In embodiments where one or more cells are used in the reaction site, the cells may be any cell or cell type. For example, the cell may be a bacterium or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In certain embodiments, the cell may be a Chinese hamster ovarian (“CHO”) cell or a 3T3 cell. In some embodiments, more than one cell type may be used simultaneously, for example, fibroblasts and hepatocytes. In certain embodiments, cell monolayers, tissue cultures or cellular constructs (e.g., cells located on a non-living scaffold), and the like may also be used in the reaction site. The precise environmental conditions necessary in the reaction site for a specific cell type or types may be determined by those of ordinary skill in the art.

In some instances, the cells may produce chemical or biological compounds of therapeutic and/or diagnostic interest. For example, the cells may be able to produce products such as monoclonal antibodies, proteins such as recombinant proteins, amino acids, hormones, vitamins, drug or pharmaceuticals, other therapeutic molecules, artificial chemicals, polymers, tracers such as GFP (“green fluorescent protein”) or luciferase, etc. In one set of embodiments, the cells may be used for drug discovery and/or drug developmental purposes. For instance, the cells may be exposed to an agent suspected of interacting with the cells. Non-limiting examples of such agents include a carcinogenic or mutagenic compound, a synthetic compound, a hormone or hormone analog, a vitamin, a tracer, a drug or a pharmaceutical, a virus, a prion, a bacteria, etc. For example, in one embodiment, the invention may be used in automating cell culture to enable high-throughput processing of monoclonal antibodies and/or other compounds of interest.

In certain cases, a reactor and/or a reaction site within a chip may be constructed and arranged to prevent, facilitate, and/or determine a chemical or a biochemical reaction with the living cells within the reaction site (for example, to determine the effect, if any, of an agent such as a drug, a hormone, a vitamin, an antibiotic, an enzyme, an antibody, a protein, a carbohydrate, etc. on a living cell). For example, one or more agents suspected of being able to interact with a cell may be added to a reactor and/or a reaction site containing the cell, and the response of the cell to the agent(s) may be determined, using the systems and methods of the invention.

In some embodiments, the chip is constructed and arranged such that cells within the chip can be maintained in a metabolically active state, for example, such that the cells are able to grow and divide. For instance, the chip may be constructed such that one or more additional surfaces can be added to the reaction site, for example, as in a series of plates, or the chip may be constructed such that the cells are able to divide while remaining attached to a substrate. In some cases, the chip may be constructed such that cells may be harvested or removed from the chip, for example, through an outlet of the chip, or by removal of a surface from the reaction site, optionally without substantially disturbing other cells present within the chip. The chip may be able to maintain the cells in a metabolically active state for any suitable length of time, for example, 1 day, 1 week, 30 days, 60 days, 90 days, 1 year, or indefinitely in some cases.

In one set of embodiments, the chip is able to control an environmental factor associated with a reaction site by transporting an agent into or proximate the reaction site. Control of the delivery of the agent to the reaction site, in certain instances, may be used to control the environmental factor. In some cases, the chip is able to control the environmental factor without directly contacting the reaction site to an external or unsterilized agent, such as a liquid. As used herein, an “environmental factor” is a detectable or measurable condition of the environment associated with the reaction site, such as pH or the concentration of a compound. The factor or condition may be one located within the reaction site, and/or at a location relative to the reaction site (e.g., upstream or downstream) such that the environment within the reaction site is known or controlled. For example, the environmental factor may be an aggregate quantity, such as molarity, osmolarity, salinity, total ion concentration, pH, or color. The concentration may also be the concentration of one or more compounds present within and/or associated with the reaction site, for example, an ion concentration such as sodium, potassium, calcium, iron or chloride ions; or a concentration of a biologically active compound, such as a protein, a lipid, or a carbohydrate source (e.g., a sugar) such as glucose, glutamine, pyruvate, apatite, an amino acid or an oligopeptide, a vitamin, a hormone, an enzyme, a protein, a growth factor, a serum, or the like. In some embodiments, the substance within the reaction site may include one or more metabolic indicators, for example, as would be found in media, or as produced as a waste products from cells.

Thus, in some cases, the environmental factor within or associated with the reaction site may be altered and/or controlled without directly contacting the reaction site to an external or unsterilized agent. In one embodiment, the chip may be constructed to allow an agent to permeate or diffuse into the reaction site. For instance, the reaction site may be defined, at least in part, by a component such as a wall or a layer of the chip, through which an agent is able to permeate. The agent may be able to alter and/or control one or more of the environmental factors within or associated with the reaction site. For instance, the component may include a membrane, such as an osmotic membrane or a semipermeable membrane (e.g., with respect to the agent) that the agent is able to permeate across. In some cases, the component may be chemically or physically inert with respect to the agent. In certain instances, a flow of agent may occur on one side of the component. In some embodiments, the flow of agent on one side of the component may occur along a meandering or non-straight pathway, for example, to increase the time of contact between the agent and the component.

A component defining some or all of the reaction site may comprise a polymer that the agent is able to permeate. For example, the polymer may include nylon, polyethylene, polypropylene, polycarbonate, polydimethylsiloxane, or copolymers or blends. In another set of embodiments, the component may include a polymer substantially impermeable to the agent being transported, but the component may be constructed or designed to allow transport of the agent to occur, for example, through a region that is porous or contains a number of channels. In another embodiment, the component may be impermeable to the agent being transported, but the component may be converted to a permeable form upon the addition of a permeabilizing agent. As used herein, “permeation” and “permeate” refer to any non-bulk transport process. A non-bulk transport generally is a transport process where substantial convection or bulk flow does not occur. For example, permeation of the agent may occur through passive diffusion, or through pores or other interstices; or transport may be facilitated or enhanced in some manner, for instance, through osmosis, electrodiffusion, electroosmosis, percolation, or through the use of a permeation-enhancing compound. In some embodiments, transport may be facilitated using an externally-applied field, such as an electrical, magnetic, or a centripetal field.

In some cases, the component may be designed to transport an agent across the component within a given period of time or under a certain condition. The exact desired thickness, density, porosity, tortuosity, composition, or other characteristics of the component may be determined by those of ordinary skill in the art. In certain cases, transport of the agent may be relatively rapid. For instance, the component may be constructed such that an agent is transported across in less than about 10 minutes, less than about 5 minutes, less than about 3 minutes, or less than about 1 minute.

In some embodiments, an environmental factor within the reaction site may be altered by generating one or more chemical agents within the chip, for example, from a precursor, that interact with, or alter in some way, an environmental factor associated with the reaction site. In one embodiment, the chemical agent may be generated within the reaction site. In another embodiment, the chemical agent may be generated elsewhere and transported to the reaction site. For example, the chemical agent may be produced and/or stored within a different compartment associated with or external of the chip (e.g., as in a reservoir), then transported to the reaction site, for instance, through a channel or other fluidic connection, or by allowing it to permeate or diffuse across a membrane or another component. In one embodiment, the agent may be generated in a location proximate the reaction site, e.g., such that it can be transferred to the reaction site, for example, in a few seconds. In another embodiment, the agent may be a gas transported to the reaction site, for example, through a membrane, or over a barrier that prevents liquid communication between the compartment and the reaction site. The reaction may be externally initiated in certain embodiments. For example, a light source, such as a laser, may be applied to the reactants, or heat may be used to initiate a reaction. In yet another embodiment, a fluidic connection may be created between the compartment and the reaction site, for example, reversibly. For instance, the fluidic connection may be created by opening a valve such as a mechanical valve or a micromechanical valve, etc. separating the compartment and the reaction site.

In some cases, additional compounds may be combined with the precursors to, for example, preserve the precursors against decomposition, to enhance the ability of the precursors to react (e.g., a catalyst or an enzyme), or to enhance the absorption of incident energy onto the chemical, for instance, to increase the chemical reaction rate. In one set of embodiments, a material that is absorptive of incident electromagnetic radiation is a darkened or “black” material which may be added to the precursors, for example, to enhance the absorption of light energy. Non-limiting examples of black material include quartz, black glass, silicon, black sand, carbon black, and the like. The additional compounds may be substantially unreactive, unable to form a transportable agent, or the additional compounds may not significantly interfere with the production of the agents or control of the environmental factors associated with the reaction site. The chemical agent, in certain embodiments, may be produced in a reaction that is activated at a certain temperature, such as in a thermal decomposition or degradation reaction. In some cases, the reaction may be initiated when the precursors are exposed to at least a certain temperature. The temperature necessary to activate the reaction may be produced, for example, upon the application of light energy, heat, an exothermic chemical reaction, or the like. In some instances, the generated chemical agent may be a gas, for example, O₂, CO, CO₂, NO, NO₂, ammonia, acetic acid, or the like. In some cases, the chemical reaction may produce one or more gases and/or one or more non-gaseous products. The gases may then be transported into the reaction site (for example, through a membrane or over a barrier), while non-gaseous products may be prevented from entering the reaction site.

Chips of the invention typically include or are connected to one or more fluid pathways for delivery of species or removal of species from a reaction site. In some cases, a fluidic pathway can be created in situ (after construction of the chip, during chip setup and/or during use of the chip) by permeabilizing or damaging a component separating the compartment from the reaction site (e.g., as in a wall or a membrane), or separates the compartment from a fluidic pathway in fluid communication with the reaction site. For example, the component may be permeabilized by heating the component to increase the permeability of the chemical agent, or by causing the component to melt or vaporize. The component, in some cases, may also be dissolved or damaged through a reaction, for example, a chemical or electrochemical reaction, to produce a fluidic connection with the reaction site. For example, the component may include a metal, such as gold, silver or copper, that can be electrolyzed upon the application of a suitable electrical current. As another example, the component may be chemically etched, for example, with a reactive species. In still other embodiments, the component may be mechanically damaged, for example, by piercing the surface with a microneedle, which may originate from within the chip, or externally. The component, may also be damaged without the use of mechanical forces or chemicals. For example, energy may be applied to the surface to damage it. In one embodiment, the component may be ablated, for example, using light. If light is used, the light may be channeled through a waveguide to the surface in some cases, or light may be applied directly to the surface. In some cases, the permeability of the component may be enhanced by one, two, or three or more orders of magnitude. In certain cases, the enhancement may be reversible, for example, by decreasing temperature, or introducing a non-permeabilizing agent. The component may include a material able to enhance the creation of the fluidic pathway in some cases. For example, the material may facilitate the absorption of light energy, or increase the chemical reaction or transport rate. For instance, in one embodiment, the surface comprises a material that is absorptive of incident electromagnetic radiation, such as quartz, black glass, silicon, black sand, carbon black, etc. As another example, the component may include a catalyst, an enzyme, or a permeation enhancer.

In some embodiments, any of the above-described chips may be constructed and arranged such that the chip is able to respond to a change in an environmental condition within a reaction site of the chip, for example, by use of a control system. Detection of the environmental condition may occur, for example, by means of a sensor which may be positioned within the reaction site, or positioned proximate to the reaction site, i.e., such that it is in communication with the reaction site (for example, fluidly, optically, thermally, pneumatically, or electronically) to the extent that it can sense one or more conditions within the reaction site. The sensor may be, for example, a pH sensor, an oxygen sensor, a sensor able to detect the concentration of a substance, or the like. The sensor may be embedded and integrally connected with the chip, or separate from the chip. The sensor may be integrally connected to or separate from the reaction site.

In one set of embodiments, the chip may include a control system. As used herein, a “control system” is a system able to detect and/or measure one or more environmental factors within or associated with the reaction site, and cause a response or a change in the environmental conditions within or associated with the reaction site (for instance, to maintain an environmental condition at a certain value). The response produced by the control system may be based on the environmental factor in certain cases. An “active” control system, as used herein, is a system able to cause a change in an environmental factor associated with a reaction site as a direct response to a measurement of the environmental condition. The active control system may provide an agent that can be delivered, or released from the reaction, where the agent is controlled in response to a sensor able to determine a condition associated with the reaction site. A “passive” control system, as used herein, is a system able to maintain or cause a change in an environmental condition of the reaction site without requiring a measurement of an environmental factor. The passive control system may control the environmental factor within the reaction site, but not necessarily in response to a sensor or a measurement. The passive control system may allow an agent to enter or exit the reaction site without active control. For example, a passive control system may include an oxygen membrane and/or a water-permeable membrane, where the membrane can maintain the oxygen and/or the water vapor content within the reaction site, for instance, within certain predetermined limits. The control system may be able to control one or more conditions within or associated with the reaction site for any length of time, for example, 1 day, 1 week, 30 days, 60 days, 90 days, 1 year, or indefinitely in some cases.

As used herein, a “processor” or a “microprocessor” is any component or device able to receive a signal from one or more sensors, store the signal, and/or convert the signal into one or more responses for one or more actuators, for example, by using a mathematical formula, or an electronic or computational circuit. The signal may be any suitable signal indicative of the environmental factor determined by the sensor, for example a pneumatic signal, an electronic signal, an optical signal, a mechanical signal, etc. The processor may be any device suitable for determining a response to the signal, for example, a mechanical device, or an electronic device such as a semiconductor chip. The processor may be embedded and integrally connected with the reaction site or chip or separate from the reaction site or chip, depending on the application. In one embodiment, the processor is programmed with a process control algorithm, which can, for example, take an incoming signal from a sensor and convert the signal into a suitable output for an actuator. Any suitable algorithm(s) may be used within the processor, for example, a PID control system, a feedback or feedforward system, a fuzzy logic control system, etc. The processor may be programmed or otherwise designed to control an environmental condition within the reaction site, for example, by manipulation of an actuator.

As used herein, an “actuator” is a device able to affect the environment within or proximate to one or more reaction sites, or in an inlet or outlet in fluid communication with one or more reaction sites. The actuator may be separate from, or integrally connected to the reaction site or chip. For example, in some embodiments, the actuator may include a valve or a pump able to control, alter, and/or prevent the flow of a substance or agent into or out of the reaction site, for example, a chemical solution, a buffering solution, a gas such as CO₂ or O₂, a nutrient solution, a saline solution, an acid, a base, a solution containing a carbon source, a nitrogen source, an inhibitor, a promoter, a hormone, a growth factor, an inducer, etc. The substance to be transported will depend on the specific application.

In some cases, the pump may be external of the chip. As one example, the actuator may selectively open a valve that allows CO₂ or O₂ to enter the reaction site. In other cases, the pump may be internal of the chip. For example, the pump may be a piezoelectric pump or a mechanically-activated pump (e.g., activated by pressure, electrical stimulation, etc.). In one embodiment, the pump is activated by producing a gas within the chip, which may cause fluid flow within the chip; as examples, the gas may be produced by directing light such as laser light at a reactant to start a gas-producing reaction, or the gas may be produced by applying an electric current to a reactant (for instance, an electric current may be applied to water to produce gas). As another example, the actuator may include a pumping system that can create a fluid connection with a reaction site as necessary.

In one aspect, the present invention provides any of the above-mentioned chips packaged in kits, optionally including instructions for use of the chips. That is, the kit can include a description of use of the chip, for example, for use with a microplate, or an apparatus adapted to handle microplates. As used herein, “instructions” can define a component of instruction and/or promotion, and typically involve written instructions on or associated with packaging of the invention. Instructions also can include any oral or electronic instructions provided in any manner such that a user of the chip will clearly recognize that the instructions are to be associated with the chip. Additionally, the kit may include other components depending on the specific application, for example, containers, adapters, syringes, needles, replacement parts, etc. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention.

The function and advantage of these and other embodiments of the present invention will be more fully understood from the examples below. The following examples are intended to illustrate the benefits of the present invention, but do not exemplify the full scope of the invention.

### EXAMPLE

In this example, an embodiment of the present invention is illustrated as used in a chip sealed by a membrane having a permeability to oxygen high enough to allow culture of living cells. The amount of oxygen required in this example is a function of the number of cells present and the oxygen requirements for the cells' metabolism. This is illustrated in the equations 1-3 below.

$$\frac{P A ( {p}_{i n} - {p}_{out} )}{l} = \frac{Δ {m}_{gas}}{Δ t} = n r V \qquad ( 1 )$$

V=Ad (2)

$$P = \frac{n r d l}{{p}_{i n} - {p}_{out}} \qquad ( 3 )$$

In these equations, P represents the permeability (typically measured in units of cm³_{STP} mm/m² atm day), A is the area (typically measured in m²), pᵢₙ is the oxygen partial pressure in the chip (typically measured in atm), pₒᵤₜ is the oxygen partial pressure outside the chip (typically measured in atm), l is the membrane thickness (typically measured in micrometers), V is the volume of the chip (typically measured in microliters), d is the cell culture chamber depth (typically measured in micrometers), n is the cell density (typically measured in cell/ml), and r is the specific oxygen demand per cell (typically measured in O₂/cell h).

Equation 1 represents a mass balance equating oxygen consumed by the growing culture to that available via diffusion through the film. Equation 2 sets the volume of the culture chamber equal to cross sectional area of the membrane contacting the chamber equal area out of both sides. Rearrangement yields Equation 3, thus expressing the minimum oxygen permeability needed to sustain cells of a given population density and metabolic rate as a function of film thickness and chamber depth

Values for P generally depend on the polymer and the permeant system, and were varied in this example for oxygen between 39,000 (cm³_{STP} mm/m² atm day) for silicon to 0.01 (cm³_{STP} mm/m² atm day) for EVA; pᵢₙ was varied between 0.05 atm and 0.2 atm, and pₒᵤₜ was assumed to be 0.2 atm. The film thickness, 1, was varied between 1 micrometer and 2 mm. V was held to be less than 1 ml, and the cell culture depth, d, ranged between 30 micrometers and 2 mm. The cell density, n, was assumed in this example to be between 10⁵ cells/ml and 10⁷ cells/ml for mammalian cells and between 10⁹ cells/ml and 10¹¹ cells/ml for bacteria. The specific oxygen demand per cell ranged between 0.5 and 5×10⁻¹² mol O₂/cell h.

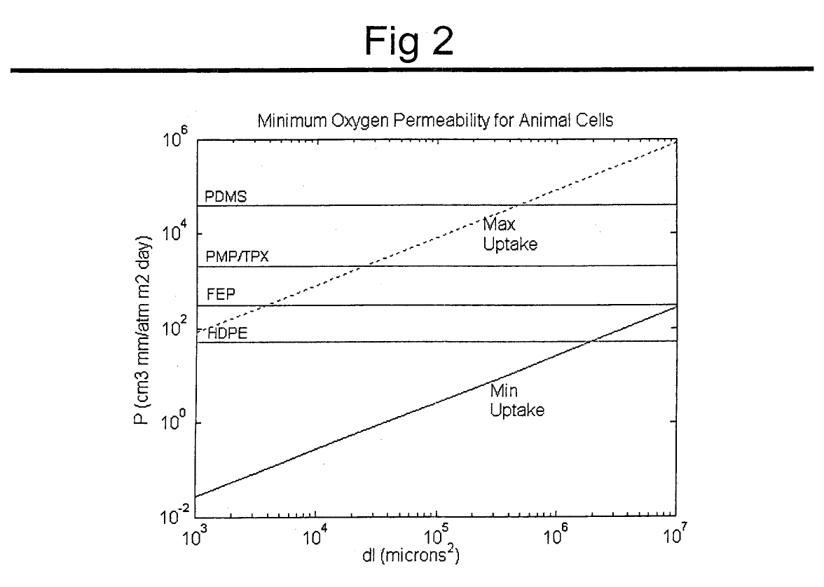

Equations 1-3 were then used to generate FIG. 1 and FIG. 2. FIG. 1 is a graph of oxygen permeability requirements for bacterial cell culture as a function of film thickness and device geometry. FIG. 2 is a graph of oxygen permeability requirements for bacterial cell culture as a function of film thickness and device geometry. In both figures, flat horizontal lines represent the permeability of likely membrane or thin film construction materials, while diagonal lines represent the highest and lowest expected oxygen requirement. In these figures, n, the cell density, and r, the specific reaction rate, were set to the highest and lowest values, and the partial pressure differential (pᵢₙ−pₒᵤₜ) was set to 0.05 atm. The required permeability was then linear in the product of d, the chip depth and l, the thickness of the covering film.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A membrane comprising a material having a permeability to oxygen greater than about 50 (cm³_{STP} mm/m² atm day) and a permeability to water vapor lower than about 6×10⁶ (cm³_{STP} mm/m² atm day).

2. The membrane of claim 1, wherein the membrane is translucent or substantially translucent.

3. The membrane of claim 1, wherein the membrane is transparent or substantially transparent.

4. The membrane of claim 1, wherein the membrane is between 10 micrometers and 2 millimeters thick.

5. A membrane comprising:
a first layer comprising at least 55% by weight of a first polymer or copolymer;
a second layer comprising no more than 45% by weight of the first polymer or copolymer;
a permeability to oxygen greater than about 1×10² (cm³_{STP} mm/m² atm day); and
a permeability to water vapor lower than about 6×10⁶ (cm³_{STP} mm/m² atm day).

6. The membrane of claim 5, wherein the permeability to oxygen is greater than about 1×10³ (cm³_{STP} mm/m² atm day).

7. The membrane of claim 5, wherein the permeability to water vapor lower than about 1×10⁵ (cm³_{STP} mm/m² atm day).

8. The membrane of claim 5, wherein the first and second layers are laminated together.

9. The membrane of claim 5, wherein the first and second layers are at least partially intermixed.

10. The membrane of claim 5, wherein a portion of the first layer is co-polymerized with a portion of the second layer.

11. The membrane of claim 5, wherein the first layer has a higher permeability to oxygen and water vapor than the second layer.

12. The membrane of claim 11, wherein the first layer has a permeability to oxygen between about 1.0×10¹ (cm³_{STP} mm/m² atm day) and about 1.0×10⁴ (cm³_{STP} mm/m² atm day) and a permeability to water vapor between about 1.0×10² (cm³_{STP} mm/m² atm day) and about 5×10⁴ (cm³_{STP} mm/m² atm day).

13. The membrane of claim 11, wherein the second layer has a permeability to oxygen between about 1×10⁴ (cm³_{STP} mm/m² atm day) and about 1×10⁵ (cm³_{STP} mm/m² atm day) and a permeability to water vapor between about 1×10⁵ (cm³_{STP} mm/m² atm day) and about 1×10⁷ (cm³_{STP} mm/m² atm day).

14. The membrane of claim 11, wherein the first layer has a permeability to oxygen between about 1×10¹ (cm³_{STP} mm/m² atm day) and about 1×10⁴ (cm³_{STP} mm/m² atm day) and a permeability to water vapor between about 1×10² (cm³_{STP} mm/m² atm day) and about 1×10⁶ (cm³_{STP} mm/m² atm day) and the second layer has a permeability to oxygen between about 1×10⁴ (cm³_{STP} mm/m² atm day) and about 1×10⁵ (cm³_{STP} mm/m² atm day) and a permeability to water vapor between about 1×10⁵ (cm³_{STP} mm/m² atm day) and about 1×10⁷ (cm³_{STP} mm/m² atm day).

15. The membrane of claim 5, wherein the first layer comprises a material selected from the group consisting of polydimethlysiloxane, a polyfluoroorganic material, polystyrene, HDPE, LDPE, LLDPE, ULDPE, poly(4-methyl pentene-1), poly(1-trimethylsilyl-1-propyne), and combinations, analogs, and derivatives thereof.

16. The membrane of claim 5, wherein the first layer comprises poly(4-methyl pentene-1).

17. The membrane of claim 16, wherein the first layer comprises at least 50% poly(4-methyl pentene-1) by weight.

18. The membrane of claim 5, wherein the first layer comprises poly(1-trimethylsilyl-1-propyne).

19. The membrane of claim 16, wherein the first layer comprises at least 50% poly(1-trimethylsilyl-1-propyne) by weight.

20. The membrane of claim 5, wherein the first layer comprises at least 50% polyfluoroorganic material by weight.

21. The membrane of claim 5, wherein the first layer is between about 10 micrometers and 2 millimeters thick.

22. The membrane of claim 21, wherein the second layer is between about 10 micrometers and 2 millimeters thick.

23. The membrane of claim 5, wherein the membrane is translucent or substantially translucent.

24. The membrane of claim 5, wherein the membrane is transparent or substantially transparent.

25. The membrane of claim 5, wherein the membrane is between 10 micrometers and 2 millimeters thick.

26. An apparatus, comprising:
a chip comprising a predetermined reaction site including a membrane comprising a permeability to oxygen greater than about 1×10² (cm³_{STP} mm/m² atm day); and
a permeability to water vapor lower than about 6×10⁶ (cm³_{STP} mm/m² atm day).

27. The apparatus of claim 26, wherein the chip is constructed and arranged to maintain at least one living cell at the predetermined reaction site.

28. The apparatus of claim 26, wherein the membrane includes a cell adhesion layer.

29. The apparatus of claim 28, wherein cell adhesion layer comprises a material selected from the group consisting of polyfluoroorganic materials, polyester, polydimethlysiloxane, polycarbonate, polystyrene, poly(4-methyl pentene-1), poly(1-trimethylsilyl-1-propyne), aluminum oxide, and combinations thereof.

30. The apparatus of claim 28, wherein the cell adhesion layer comprises a modified surface.

31. The apparatus of claim 28, wherein the cell adhesion layer is an inner layer of the membrane and abuts an interior of the predetermined reaction site.

32. The apparatus of claim 26, wherein the membrane comprises a support layer.

33. The apparatus of claim 32, wherein the support layer is one of an outer layer and an intermediate layer.

34. The apparatus of claim 32, wherein the support layer comprises a material selected from a group consisting of polydimethylsiloxane, latex, glass, silicon, polystyrene, polyester, poly(4-methyl pentene-1), poly(1-trimethylsilyl-1-propyne), and combinations thereof.

35. The apparatus of claim 26, further comprising a predetermined reaction site no greater than 1 millimeter in maximum cross section, wherein the membrane abuts the predetermined reaction site.

36. The apparatus of claim 26, further comprising a predetermined reaction site no greater than 20 cm² in maximum cross sectional area, wherein the membrane abuts the predetermined reaction site.

37. The apparatus of claim 26, further comprising at least 7 predetermined reaction sites, and wherein the membrane abuts at least one of the at least 7 predetermined reaction sites.

38. The apparatus of claim 26, further comprising at least 20 predetermined reaction sites, and wherein the membrane abuts at least one of the at least 20 predetermined reaction sites.

39. An apparatus, comprising:
a chip comprising a predetermined reaction site no greater than 1 milliliter in volume; and
a humidity controller positioned adjacent to the predetermined reaction site.

40. The apparatus of claim 39, wherein the chip is constructed and arranged to maintain at least one living cell at the predetermined reaction site.

41. The apparatus of claim 39, wherein the humidity controller has high enough oxygen permeability and low enough water vapor permeability to allow cell growth within the predetermined reaction site.

42. The apparatus of claim 39, wherein the cell growth is mammalian cell growth.

43. The apparatus of claim 39, wherein the cell growth is animal cell growth.

44. The apparatus of claim 39, wherein the humidity controller has a permeability to oxygen greater than about 1×10² (cm³_{STP} mm/m² μm day) and a permeability to water vapor lower than about 6×10⁶ (cm³_{STP} mm/m² atm day).

45. The apparatus of claim 39, wherein the humidity controller is positioned in a wall of the predetermined reaction site.

46. The apparatus of claim 39, wherein the humidity controller comprises a membrane.

47. The apparatus of claim 39, wherein the humidity controller comprises a cell adhesion layer.

48. The apparatus of claim 47, wherein the cell adhesion layer is positioned in an inner wall of the predetermined reaction site.

49. A method of culturing cells, comprising:
identifying an oxygen requirement and a humidity requirement of the cells;
selecting a material having an oxygen permeability high enough to meet the oxygen requirement of the cells and a water vapor permeability low enough to meet the humidity requirement of the cells; and
culturing the cells in a chip comprising a predetermined reaction site having a volume of no more than 1 milliliter and at least one wall having at least a portion thereof formed of the material.

50. The method of claim 49, further comprising culturing the cells for at least 24 hours.

51. The method of claim 49, further comprising culturing the cells for at least 48 hours.

52. The method of claim 49, further comprising culturing the cells for at least 1 week.

53. The method of claim 49, further comprising culturing the cells for at least 2 weeks.

54. The method of claim 49, further comprising culturing the cells for at least 4 weeks.

55. The method of claim 49, further comprising culturing the cells for at least 6 weeks.

56. The method of claim 49, further comprising observing the cells through the material.

57. The method of claim 49, wherein, during culturing, the humidity external to the chip is insufficient to meet the humidity requirement of the cells.

58. An apparatus, comprising:
a chip comprising a predetermined reaction site no greater than 1 milliliter in volume; and
a humidity controller having sufficient oxygen permeability and a low water vapor permeability selected to allow cell growth within the predetermined reaction site.

59. The apparatus of claim 58, wherein the chip is constructed and arranged to maintain at least one living cell at the predetermined reaction site.

60. The apparatus of claim 58, wherein the humidity controller is a membrane.

61. The apparatus of claim 58, wherein the humidity controller is a thin film.

62. An apparatus, comprising:
a chip comprising a predetermined reaction site no greater than 1 milliliter in volume; and
a humidity controller able to maintain a humidity level and an oxygen concentration in the predetermined reaction site sufficient to allow cell growth within the predetermined reaction site.

63. The apparatus of claim 62, wherein the chip is constructed and arranged to maintain at least one living cell at the predetermined reaction site.

64. The apparatus of claim 62, wherein the humidity controller is a membrane.

65. The apparatus of claim 62, wherein the humidity controller is a thin film.

66. An apparatus, comprising:
a chip comprising a predetermined reaction site no greater than 1 milliliter in volume; and
a humidity controller positioned adjacent to the predetermined reaction site.

67. The apparatus of claim 66, wherein the chip is constructed and arranged to maintain at least one living cell at the predetermined reaction site.

68. An apparatus, comprising:
a chip comprising a predetermined reaction site no greater than 1 milliliter in volume; and
a humidity controller having sufficient oxygen permeability and a low water vapor permeability selected to allow cell growth within the predetermined reaction site.

69. The apparatus of claim 68, wherein the chip is constructed and arranged to maintain at least one living cell at the predetermined reaction site.

70. An apparatus, comprising:
a chip comprising a predetermined reaction site no greater than 1 milliliter in volume; and
a gas-permeable surface positioned adjacent to the predetermined reaction site.

71. The apparatus of claim 70, wherein the chip is constructed and arranged to maintain at least one living cell at the predetermined reaction site.

72. A method, comprising:
diffusing a gas into a predetermined reaction site no greater than 1 milliliter in volume.
